SEQUENCE LISTING (41 sequences)

SEQ ID NO: 1 - DNA, 10 bp, Artificial Sequence
  RHO-ADRP
gcaggcctct

SEQ ID NO: 2 - DNA, 24 bp, Artificial Sequence
  CRX
ctccgtgggaccttccctggcccc

SEQ ID NO: 3 - DNA, 21 bp, Rattus norvegicus
ggcgtggtgcgcagccccttt

SEQ ID NO: 4 - DNA, 21 bp, Rattus norvegicus
acccattaagctgctgcttcc

SEQ ID NO: 5 - DNA, 21 bp, Rattus norvegicus
ggcgtggtgcgcagccccttt

SEQ ID NO: 6 - DNA, 21 bp, Rattus norvegicus
acccattaagctgctgcttcc

SEQ ID NO: 7 - DNA, 22 bp, Rattus norvegicus
caaaaggccaaactgccctaag

SEQ ID NO: 8 - DNA, 22 bp, Rattus norvegicus
atttggctgcgtctgtagatga

SEQ ID NO: 9 - DNA, 23 bp, Rattus norvegicus
tggttgttgggtactggtagatg

SEQ ID NO: 10 - DNA, 20 bp, Rattus norvegicus
ggcgcgcatagttgcagttt

SEQ ID NO: 11 - DNA, 20 bp, Rattus norvegicus
cttggccgaggtacttctca

SEQ ID NO: 12 - DNA, 23 bp, Rattus norvegicus
ttcagtttcctgatggcaatgta

SEQ ID NO: 13 - DNA, 24 bp, Rattus norvegicus
actcaattccccaataaaaaggca

SEQ ID NO: 14 - DNA, 20 bp, Rattus norvegicus
tgtgaggtccaacatgtgct

SEQ ID NO: 15 - DNA, 19 bp, Rattus norvegicus
tccacgaacagccaacctt

SEQ ID NO: 16 - DNA, 22 bp, Rattus norvegicus
gttgccagcctagataacctca

SEQ ID NO: 17 - DNA, 22 bp, Rattus norvegicus
cctcctatgtagaactggctca

SEQ ID NO: 18 - DNA, 21 bp, Rattus norvegicus
ggtggatcacctatctggttc

SEQ ID NO: 19 - DNA, 20 bp, Rattus norvegicus
gcttctctctggttgtggct

SEQ ID NO: 20 - DNA, 20 bp, Rattus norvegicus
aggctgctgctccagatttt

SEQ ID NO: 21 - DNA, 20 bp, Rattus norvegicus
ggctggtgatggtggtcatt

SEQ ID NO: 22 - DNA, 20 bp, Rattus norvegicus
aacagtgtcttccagctgcc

SEQ ID NO: 23 - DNA, 21 bp, Rattus norvegicus
aacttcctcacgctctacgtc

SEQ ID NO: 24 - DNA, 20 bp, Rattus norvegicus
ccaatgaataagctgggcct

SEQ ID NO: 25 - DNA, 21 bp, Rattus norvegicus
aacttcctcacgctctacgtc

SEQ ID NO: 26 - DNA, 20 bp, Rattus norvegicus
ccaatgaataagctgggcct

SEQ ID NO: 27 - DNA, 19 bp, Artificial Sequence
  gRNA
tcaagtgatagtctgccat

SEQ ID NO: 28 - DNA, 24 bp, Artificial Sequence
  Target Site
agttcactatcagacggtanggat

SEQ ID NO: 29 - DNA, 24 bp, Artificial Sequence
  Target Site
tcaagtgatagtctgccattacta

SEQ ID NO: 30 - DNA, 24 bp, Artificial Sequence
  gRNA
caccgcccaaagacgaagtagcca

SEQ ID NO: 31 - DNA, 40 bp, Artificial Sequence
  Mouse target site
tagtgaggtaccgatgaagcagaaacccgggtgtccgaca

SEQ ID NO: 32 - DNA, 40 bp, Artificial Sequence
  Rat target site
gagtgacgtaccgatgaaacagaaacccgggtgtccgacg

SEQ ID NO: 33 - DNA, 36 bp, Artificial Sequence
  Rat RhoS334
tgtgggcccaagacgaagtagccatggagtgatgtg

SEQ ID NO: 34 - DNA, 37 bp, Artificial Sequence
  RhoWT
tgtgggcccaaagacaaagtagccatgcagtgaggtg

SEQ ID NO: 35 - DNA, 22 bp, Artificial Sequence
  R1
gcccaaagacgaagtaccatgg

SEQ ID NO: 36 - DNA, 19 bp, Artificial Sequence
  R2
gcccaaagacgaaccatgg

SEQ ID NO: 37 - DNA, 24 bp, Artificial Sequence
  R3
gcccaaagacgaagtaggccatgg

SEQ ID NO: 38 - DNA, 12 bp, Artificial Sequence
  R4
gcccaaccatgg

SEQ ID NO: 39 - protein, 27 aa, Homo sapiens
CCQKNPLQDDDASATASKTETSQVAPA

SEQ ID NO: 40 - protein, 12 aa, Homo sapiens
CCQKNPLQDDDA

SEQ ID NO: 41 - protein, 27 aa, Homo sapiens
CCQKNPLQDDDASATVAKTETSQVAPA

(19) United States
(12) Patent Application Publication
(10) Pub. No.: US 2016/0324987 A1
(43) Pub. Date: Nov. 10, 2016

(54) USE OF CRISPR/CAS9 AS IN VIVO GENE THERAPY TO GENERATE TARGETED GENOMIC DISRUPTIONS IN GENES BEARING DOMINANT MUTATIONS FOR RETINITIS PIGMENTOSA

(71) Applicant: Cedars-Sinai Medical Center, Los Angeles, CA (US)

(72) Inventors: Shaomei Wang, Los Angeles, CA (US); Benjamin Bakondi, Walnut Creek, CA (US); Wenjian Lv, Los Angeles, CA (US); Bin Lu, Los Angeles, CA (US)

(73) Assignee: Cedars-Sinai Medical Center, Los Angeles, CA (US)

(21) Appl. No.: 15/130,846

(22) Filed: Apr. 15, 2016

Related U.S. Application Data
(60) Provisional application No. 62/147,981, filed on Apr. 15, 2015.
(60) Provisional application No. 62/149,468, filed on Apr. 17, 2015.

(51) Int. Cl. A61K 48/00; C12N 15/86; C12N 15/90; C07K 14/705; A61K 9/00; A61K 38/17

(52) U.S. Cl. CPC A61K 48/0058; A61K 9/0019; A61K 9/0048; A61K 48/0075; A61K 38/177; C12N 15/907; C07K 14/705; C12N 15/86; C12N 2800/80; C12N 2750/14171; C12N 2750/14143

(57) ABSTRACT

Described herein are methods and compositions for genomic editing. Clustered regularly interspaced short palindromic (CRISPR) allows for highly selective targeting and alteration of genetic loci. Here, the Inventors demonstrate CRISPR as capable of being used in living animals to prophylactically prevent a genetic disease from manifesting. Targeting and disruption of mutated rhodopsin gene prevents progression of retinitis pigmentosa in the retinal cells of a transgenic rat model. Such techniques allow for treatment methods in subjects with dominant genetic mutations, often associated with lack of a gene product, or a toxic gene product. The described technology effectively abrogates deleterious effects due to the presence of a mutated gene copy allowing the normal function of the wild-type protein to prevent cell and vision loss. The efficacy of these in vivo mechanisms are widely extensible to similar dominant negative gene mutations causing disease, or other types of genetic disease.

### CROSS REFERENCE TO RELATED APPLICATIONS

This application claims the benefit of U.S. Provisional Application No. 62/147,981 filed Apr. 15, 2015 and 62/149,468 filed Apr. 17, 2015.

### GOVERNMENT RIGHTS CLAUSE

This invention was made with government support under Contract No. EY02048 awarded by the National Institutes of Health. The government has certain rights in the invention.

### FIELD OF THE INVENTION

Described herein are methods and compositions that find use in the field of medicine as providing in vitro and in vivo manipulation of genetic sequences for research and therapeutic activities related to genetic abnormalities, such as dominant negative diseases including forms of retinitis pigmentosa.

### BACKGROUND

Retinitis pigmentosa (RP) is a genetic degenerative eye disease resulting in severe vision impairment and blindness due to degeneration of the rod photoreceptor cells in the retina. This particular form of retinal dystrophy manifests itself beginning with compromised peripheral and dim light vision due to the decline of the rod photoreceptors. Further rod degeneration is coupled with later abnormalities in adjacent retinal pigment epithelium (RPE) and the deterioration of cone photoreceptor cells. As peripheral vision becomes increasingly compromised, patients experience progressive “tunnel vision” and eventual blindness. Affected individuals acquire initial symptoms independent of age and diagnosis occurs anywhere from early infancy to late adulthood, and may further include defective light-dark adaptations, night blindness, and the accumulation of bone spicules in the eye.

Underlying this disease, RP is one of the most common forms of inherited retinal degeneration and multiple genes, when mutated, can cause the RP phenotype. Among these, include mutations of the gene for rhodopsin, a pigment that plays a critical role in the visual transduction cascade ordinarily providing for vision, including low-light conditions. Many such mutations in rhodopsin are missense mutations causing negative function that is inherited mostly in a dominant manner. This includes up to 50% of patients with RP, which is 1 in 8,000 Americans. As of now, the number of these mutations that may be treatable with the conventional technology (e.g., gene therapy) is approximately 5%.

A promising technology for the correction of disease caused by genetic mutations involves used of Clustered Regularly interspaced Short Palindromic Repeats (CRISPR) and CRISPR associated protein (cas protein, such as cas9). This gene editing technology allows for the insertion of targeted breaks in genomic DNA at very specific sites, as selected based on a particular DNA sequence. A variety of in vitro uses for CRISPR has been demonstrated including alteration of cells of various kinds such as bacteria, zebrafish, C. elegans, among many others, providing valuable insights into developmental mechanisms. However, only a handful of attempts have been made to develop CRISPR as an in vivo deliverable therapy to treat subjects afflicted with a genetic disease.

Described herein are compositions and methods for targeting and deleting production of a mutated copy of a gene in an RP animal model of progressive blindness. Administration of CRISPR-protein with guide RNA (gRNA) targeting mutant rhodopsin constructs prevented degeneration of photoreceptors in animals receiving therapy compared to control. The described compositions and methods, allowing for in vivo treatment of RP, readily extendible to various diseases that are caused by dominant mutations, including inherited diseases of other organs and systems, including for example, targeting of mutated genes for disruption/deletion, such as the SOD1 gene in amyotrophic lateral sclerosis (ALS).

### SUMMARY OF THE INVENTION

Described herein is a method of treatment comprising providing a quantity of one or more therapeutic vectors, and administering the one or more therapeutic vectors to a mammal afflicted with a disease and/or condition, wherein in vivo expression of the one or more therapeutic vector is capable of treating the mammal for the disease and/or condition. In other embodiments, the one or more therapeutic vectors, each encode at least one clustered regularly interspaced short palindromic (CRISPR) protein and one or more guide RNAs (gRNAs). In other embodiments, the CRISPR protein is cas9. In other embodiments, the one or more gRNAs comprise a sequence cognate to a target polynucleotide sequence and capable of binding to a protospacer adjacent motif (“PAM”). In other embodiments, the PAM includes the sequence NGG or NNGRRT. In other embodiments, the disease and/or condition includes a dominant mutation. In other embodiments, the disease and/or condition comprising a dominant mutation is retinitis pigmentosa (RP). In other embodiments, the RP includes a mutation in rhodopsin (RHO). In other embodiments, the mammal includes a human. In other embodiments, the therapeutic vector includes an adenovirus, adeno associated virus or lentivirus. In other embodiments, administering the one or more therapeutic vectors includes subretinal injection. In other embodiments, treating the mammal for the disease and/or condition includes in vivo generation of a double stranded break in a population of cells in the mammal. In other embodiments, the methods includes providing a quantity of DNA template and co-administering the DNA template. In other embodiments, the disease and/or condition includes a recessive mutation.

Also described herein is an in vivo method of genomic editing comprising providing a quantity of one or more vectors each encoding at least one clustered regularly interspaced short palindromic (CRISPR) protein and one or more guide RNAs (gRNAs), and administering the one or more vectors to a mammal, wherein in vivo expression of the one or more vectors includes binding of the CRISPR protein to a locus cognate to the gRNA and in vivo generation of a double stranded break (DSB) in a population of cells in the mammal, wherein in vivo homologous recombination (HR) of the DSB results in editing of the genome of a population of cells in the mammal. In other embodiments, the CRISPR protein is cas9 and the one or more gRNAs comprise a sequence capable of binding to a protospacer adjacent motif (“PAM”). In other embodiments, HR includes non-homologous end joining (NHEJ) introducing missense or nonsense of a protein expressed at the locus. In other embodiments, HR includes homology directed repair (HDR) introducing template DNA co-administered in step. In other embodiments, HR corrects a dominant mutation. In other embodiments, HR corrects a recessive mutation. In other embodiments, the vector includes an adenovirus, adeno associated virus or lentivirus. In other embodiments, the dominant mutation includes a mutation in rhodopsin (RHO), the mammal includes a human, and administering the one or more vectors includes subretinal injection.

Further described herein is a composition comprising a vector encoding a clustered regularly interspaced short palindromic (CRISPR) protein and one or more guide RNAs (gRNAs), wherein the one or more gRNAs comprise a sequence cognate to a target polynucleotide sequence and capable of binding to a protospacer adjacent motif (“PAM”). In other embodiments, the CRISPR protein is cas9 and the gRNA is cognate to a locus encoding rhodopsin (RHO).

### BRIEF DESCRIPTION OF FIGURES

FIG. 1. Various methods related to the technology including (FIG. 1A) Construct design (FIG. 1B) gRNA design (FIG. 1C) plasmid subretinal injected (FIG. 1D) side view of injection site in the eye (FIG. 1E) microscopic evaluation of cells and (FIG. 1F) electrical functional activation.

FIG. 2. The S334ter transgenic rat model of retinal degeneration. Mouse and rats that serve as models for RHO adRP mutations; Specifically as models for C-terminal RHO truncation mutations (e.g. Gln344ter). FIG. 2A) A base-pair mutation in exon 5 results in the substitution of serine (S) at amino acid (a.a.) position 334 for an early termination codon (ter), resulting in the production of a dominant-negative rhodopsin isoform truncated by the last 15 C-terminal a.a. residues (RhoS334ter). FIG. 2B These 15 a.a.'s contain three serines (S) that are phosphorylated for rhodopsin de-activation following light stimulation and are required for proper sub-cellular localization. FIG. 2C S334ter transgenic rats were generated using the 11 kb BamHI genomic fragment in Sprague-Dawley rats (SD-Tg(S334ter)3LavRrrc. FIG. 2D The resulting pathologic and phenotypic characteristics include the rapid and progressive degeneration of light-sensing photoreceptor cells from 10-12 rows of in the outer nuclear layer (ONL) prior to postnatal day (P) 11 to 1-2 rows at P30. Antibodies specific to the Nterminalregion of rhodopsin recognize both native (rat; FIG. 2E & FIG. 2G) and transgene (mouse, FIG. 2F & FIG. 2H) isoforms. Antibody labeling for C-terminal rhodopsin only recognizes the native (rat; FIG. 2F & FIG. 2H) isoform. Rho mislocalization is seen early in degenertation (P14; FIG. 2E & FIG. 2F) with few photoreceptors remaining by P33 (FIG. 2G & FIG. 2H).

FIG. 3. Correct genome targeting of RhoS334ter by guide-RNA. FIG. 3A) Guide-RNAs were designed complementary to DNA flanking a protospacer adjacent motif (PAM) unique to the mouse sequence to ensure selective cleavage of mutant RhoS334ter. FIG. 3B) Bone marrow-derived mesenchymal progenitor cells were isolated from S334ter-3 rats (BMSCS334ter) & transfected with plasmid containing mRNA transcripts for Cas9/gRNA/mCherryreporter.BMSCs were harvested at 4 days and genomic DNA was extracted for sequencing. FIG. 3C & FIG. 3D) Quality Scores show a decrease in nucleotide read fidelity downstream of the DNA cut site in the correct targeting gRNA (FIG. 3D) compared with incorrect targeting gRNA (FIG. 3C). This indicated the presence of multiple rhodopsin sequences among BMSCs resulting from variable endonuclease activity by Cas9 DNA disrupted prior to non-homologous end joining (NHEJ). It is noted that the mouse and rat Rho differed at the PAM site by only 1 base pair, which was pivotal to allow cleavage of only the mutant transcript. There is a also a 1 bp difference between mouse & rat Rho in the gRNA binding sequence at base position #10, which is beyond the seed region of the target DNA and is not considered critical for allele discrimination during cleavage. The 1 bp difference at the PAM site is analogous to human SNP-causing mutations, suggesting the described methodology as translatable as long as 1) the SNP creates a new PAM, or 2) one alters the PAM recognition sequence of Cas9 such that it identifies the SNP. This latter methodology render a large number of mutations targetable.

FIG. 4. Biodistribution of photoreceptor rescue correlates with mCherry expression. Animals received unilateral subretinal injections of 6 μg plasmid DNA of Rho-targeting gRNA (1 μl of 6 ug/μl) at P0 and were electroporated to facilitate plasmid uptake by proliferating photoreceptors. Five pulses of 950 mV separated by 50 ms intervals was applied via positive electrode over the injected eye. Retinas were removed between P25-75 and dissected by whole-mount procedure to determine relative biodistribution by mCherry reporter expression. FIG. 4A) 29% coverage by area at P33 is shown. The degree of photoreceptor rescue was determined from 10 μm frozen transverse sections. FIG. 4B & FIG. 4D) A single row of photoreceptors were observed in the outer nuclear layer (ONL) adjacent to plasmid injected areas. ONL thickness in correct-targeted gRNA-injected areas were markedly preserved with up to 8 rows of mCherry+ photoreceptors (FIG. 4C & FIG. 4D). FIG. 4B & FIG. 4C) Differences in the activation state of Muller glia in response to the degenerative retinal environment (GFAP staining) did not appear robust as shown in FIG. 4E.

FIG. 5. In vivo correction of retinal defects. FIG. 5A) 7 rows of PRs remained from gRNATRGT treatment compared FIG. 5B) to 1 row with control in S334 rats.ig. 5C) Upper Panel: Recoverin immunostaining distinctly localized to areas of rescued ONL, the presence of which was only observed at sites of mCherry expression. Lower Panel: Native (C-terminal) rhodopsin exclusively co-localized with mCherry+ reporter and rescued photoreceptors at P55. This confirmed that Cas9 ablated the mouse transgene (i.e. through ORF disruptions that prevented RhoS334ter transcription). Decreased mutant rho expression permitted subcellular trafficking of native (rat) Rho.

FIG. 6. Allele-specific targeting and disruption of Rho_{S334} in vitro. FIG. 6A) Schematic of px330 construct used. FIG. 6B) gRNA_{TRGT} and predicted genomic DNA binding sites in Rho_{S334} and Rho_{WT}. PAM (red underlined bases) and mismatches (red font) are shown. FIG. 6C) Phase-contrast photomicrograph of mCherry+ and mCherry− MSC_{SS334} 3-days post-lipofection with gRNA constructs prior to FACS isolation. FIG. 6D) FACS gating strategy for mCherry+ MSC_{S334} isolation is shown. mCherry+ cells represented 12% of the total population, with the brightest 3.4% mCherry+ MSC_{SS334} selected for genomic DNA sequence analysis using PCR amplicons encompassing predicted Cas9 cleavage sites. FIG. 6E) Genomic DNA sequencing results are shown with Phred quality scores (gray shading) at bottom. DNA Disruption is shown from gRNA_{TRGT}-transfected MSC_{SS334} downstream from the Cas9 cleavage site (blue highlight) in Rho_{S334} (FIG. 6E), but not in Rho_{WT} (FIG. 6F). Genomic disruption was absent at the Rho_{S334} locus using gRNA_{CNTRL} (FIG. 6G) or with no vector in untreated eyes (FIG. 6H). Scale bar=400 μm.

FIG. 7. Allele-specific targeting and disruption of Rho_{S334} in vivo. FIG. 7A) Fast-green DNA dye shows plasmid distribution following unilateral subretinal injection in S334ter-3 rats at P0. FIG. 7B) Representative retinal flat-mount shows variable mCherry intensity and uneven distribution 4 days after injection. FIG. 7C) Gating strategy for FACS-isolation of enzymatically dissociated P4 retinal cells with mCherry fluorescence intensity at high (red, 0.3%) intermediate (purple, 0.6%) and no (blue, 97.9%) expression. FIG. 7H, FIG. 7I) Sanger sequencing of PCR-amplified genomic Rho loci from PRs showed in vivo disruption of Rho_{S334} (FIG. 7D), but not Rho_{WT} (FIG. 7E) using gRNA_{TRGT}. FIG. 7F) Rho_{S334} locus targeting schematic and deep sequencing reads shows insertions/deletions (indels) proximal to the predicted cleavage site (arrowhead) from gRNA_{TRGT} expressing cells.

FIG. 8. Phenotypic rescue by Rho_{S334}-selective ablation. FIG. 8A-FIG. 8E) Fluorescent confocal images of gRNA_{TRGT} (FIG. 8A-8C) and gRNA_{CNTRL} (FIG. 8D, FIG. 8E) treated retinas at P33. FIG. 8A) Montage image shows that mCherry distribution correlated with ONL rescue (DAPI, blue) and POS formation (RHO_{WT} C-terminal immunolabel, green). Inset: Magnified image of outlined region shows preserved ONL with organized POS (arrowheads) adjoining degenerated ONL with diminished POS (arrows). FIG. 8B, FIG. 8C) RHO N-terminal (FIG. 8B) and C-terminal (FIG. 8C) immunostaining in gRNA_{TRGT}-treated retinas was absent from PR cell bodies and localized to outer segments (OS). Significant PR preservation in the ONL was observed from gRNA_{TRGT} treatment (FIG. 8B, bracket). FIG. 8D, FIG. 8E) RHO N-terminal (FIG. 8D) and C-terminal (FIG. 8E) immunostaining was absent in gRNA_{CNTRL}-treated retinas, which lacked POS formation and ONL contained one row of remaining PR nuclei (FIG. 8D, bracket).

FIG. 9. Rho_{S334} ablation preserved cone morphology and second-order retinal neuron synapses. Fluorescence confocal microscopy images from gRNA_{CNTRL} (FIG. 9A-FIG. 9D) or gRNA_{TRGT} (FIG. 9E-FIG. 9J) treated eyes at P33. FIG. 9A, FIG. 9B) Surviving PRs after gRNA_{CNTRL} treatment were non-Rho-expressing cones PRs (FIG. 9A, FIG. 9D, cone arrestin, green), which lacked typical morphological features observed in retinas rescued with gRNA_{TRGT} treatment (FIG. 9E, FIG. 9H). Individual channel images corresponding to bracketed areas in FIG. 9A and FIG. 9E show rescued PR nuclei (DAPI, FIG. 9B vs. FIG. 9F) in gRNA vector transfected areas (mCherry, FIG. 9C vs. FIG. 9G) with preserved cone morphology (i.e. pedicles and POS, FIG. 9D vs. FIG. 9H). FIG. 9I) Greater dendritic arborization of INL-resident rodbipolar neurons (PKC-α, green) was evident at the OPL in mCherry+ areas following gRNA_{CNTRL} treatment (left inset), in contrast to the adjacent degenerated area lacking mCherry+ (right inset). FIG. 9J) Similarly, synaptophysin immunolabel (green) showed greater intensity in OPL regions in which PR nuclei preservation (DAPI) corresponded with mCherry+ expression (inset: arrowheads), in sharp contrast with the adjacent unprotected area to which gRNA_{TRGT} transfection did not extend (inset: arrows). INL, Inner nuclear layer; OPL, Outer plexiform layer; PKC-α, Protein kinase C-alpha.

FIG. 10. PR rescue by gRNA_{TRGT} treatment corresponded with vision rescue. FIG. 10A) Fluorescent microscopy montage image shows mCherry+ reporter distribution (arrows) of gRNA_{TRGT} vector in a retinal flat-mount at P33 calculated at 29% of total retina area by NIH ImageJ analysis. FIG. 10B) By retinal cross-section, mCherry+ regions from gRNA_{TRGT} treatment contained significantly more PR nuclei than the mCherry+ regions from gRNA_{CNTRL} treatment, or comparable regions from untreated control areas (gRNA_{TRGT}: 307±82 PR nuclei/100 μm, N=5; vs. gRNA_{CNTRL}: 33±3, P=0.002(*), N=3; vs. Untreated: 27±13, P=0.001(†), N=4). FIG. 10C) Visual acuity (OKR) was significantly higher from gRNA_{TRGT} treatment at P39, than from gRNA_{CNTRL} treatment (gRNA_{TRGT}: 0.185±0.008 c/d, N=5, vs. gRNA_{CNTRL}: 0.121±0.009 c/d, N=4, P<0.01(†)). Visual acuity in gRNA_{TRGT}-treated eyes was significantly higher than in untreated contralateral eyes (Treated: 0.185±0.008; vs. Contralateral: 0.138±0.006 c/d, N=5, P<0.001(*)). Visual acuity in eyes injected with gRNA_{CNTRL} was not different from that of contralateral non-injected eyes (Treated: 0.121±0.009 vs. Contralateral: 0.121±0.012 c/d, N=4, P=0.763). FIG. 10D) By using the fellow eyes of individual animals as internal controls, the higher visual acuity from gRNA_{TRGT} treatment represented a 35±4.6% increase, compared to a 2.3±0.7% decrease with gRNA_{CNTRL} injection (P<0.01). c/d=cycles/degree. All values represent mean±SEM. **P≦0.01, ††P≦0.01, ***P≦0.001. N.S., not significant.

FIG. 11. PR degeneration characteristics in untreated S334ter-3 rats. S334ter-3 rats displayed standard pathologic characteristics of rapid PR degeneration in the ONL. FIG. 11A) At P14, near complete ONL thickness was observed, consisting of approximately 10 rows of DAPI+ nuclei (blue) (bracket). FIG. 11C) At P33, the ONL showed extensive degeneration from PR loss, leaving a single discontinuous row of PR nuclei (bracket). N-terminal RHO immunolabel identified both wild-type (RHO_{WT}) and mutant (RHO_{S334}) isoforms (FIG. 11A, FIG. 11C, green), while C-terminal RHO immunolabel identified only the RHO_{WT} isoform (FIG. 11B, FIG. 11D, green). At P14, RHO_{S334} was mislocalized to PR cell nuclei throughout the ONL, while RHO_{WT} was present in the ONL and polarized toward PR inner segments (FIG. 11A, 11B green).

FIG. 12. Confirmation of Rho_{S334}-specific disruption in vivo. FIG. 12A, FIG. 12B, FIG. 12C) Methodology identical to that described for FIG. 6, in which genomic DNA was extracted and sequenced from P4 retinal cells that were enzymatically dissociated and FACS-isolated into subpopulations with mCherry expression levels defined as high (mCherry_{Hi}, red), intermediate/low (mCherry_{Lo}, purple), or negative (mCherry_{Neg}, blue). Rho_{S334} allelic disruption was observed in mCherry_{Hi} and mCherry_{Lo} but not in mCherry_{Neg}, or in mCherry_{Hi} PRs at the Rho_{WT} locus (N=3 rats).

FIG. 13. Histological evaluation of phenotype rescue following Rho_{S334}-selective ablation. FIG. 13A-FIG. 13D) Light field microscopy of cresyl violet stained retinal sections at P33 from gRNA_{TRGT} treatment (FIG. 13A, FIG. 13C) and from the untreated contralateral eye from the same animal (FIG. 13B, FIG. 13D). ONL thickness is greater in the gRNA_{TRGT}-treated eye (FIG. 13A, FIG. 13C, ONL bracket) with up to 8 rows of PR nuclei compared with the 1 row of PR nuclei in the untreated eye (FIG. 13B, arrows; FIG. 13D, ONL bracket). IS were observed with gRNA_{TRGT} treatment (FIG. 13A, arrow; FIG. 13C, IS bracket), but were absent in the untreated eye (FIG. 13B, FIG. 13D). The gap between IS and RPE melanosomes (FIG. 13A, FIG. 13C, arrowheads) indicates the location of OS.

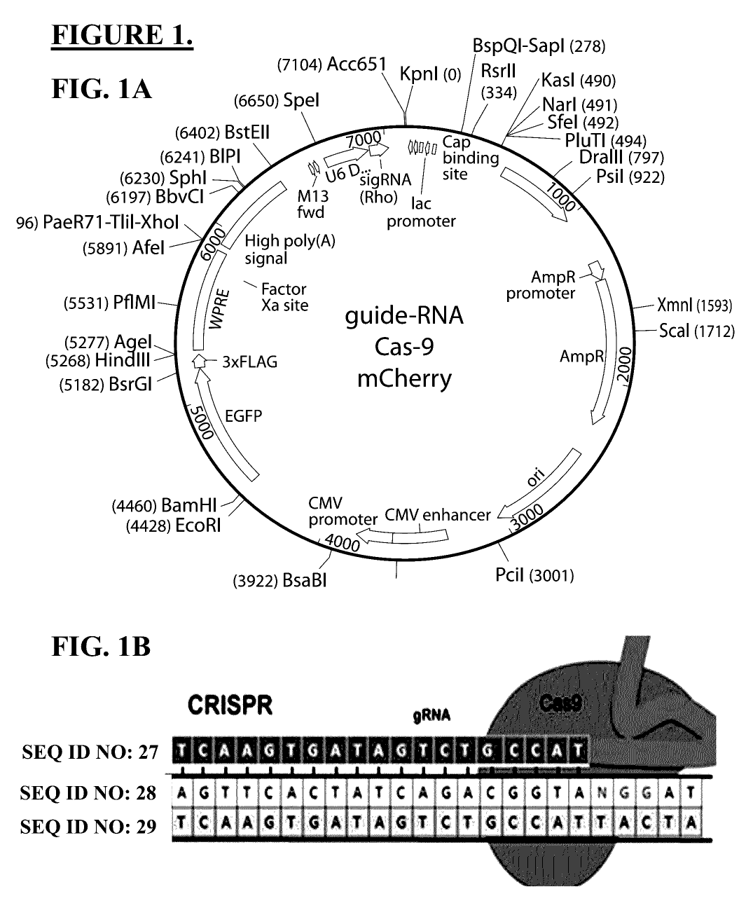

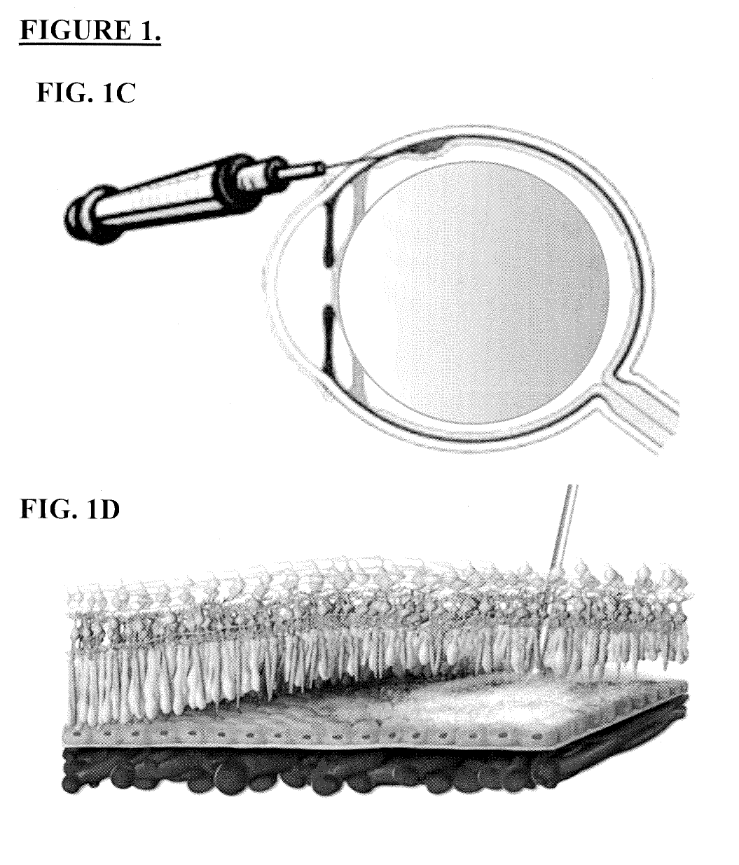

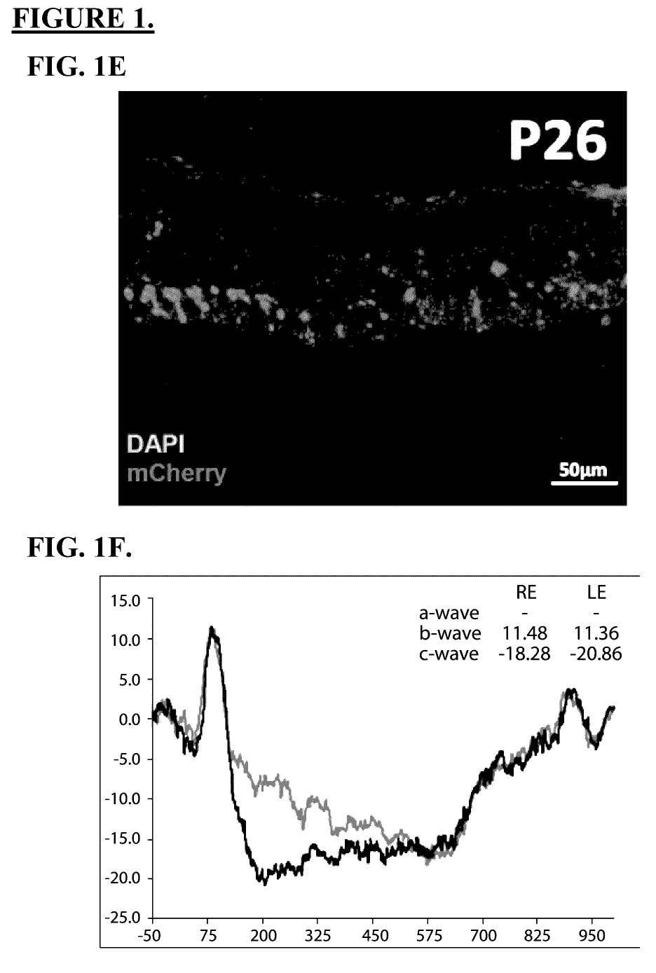

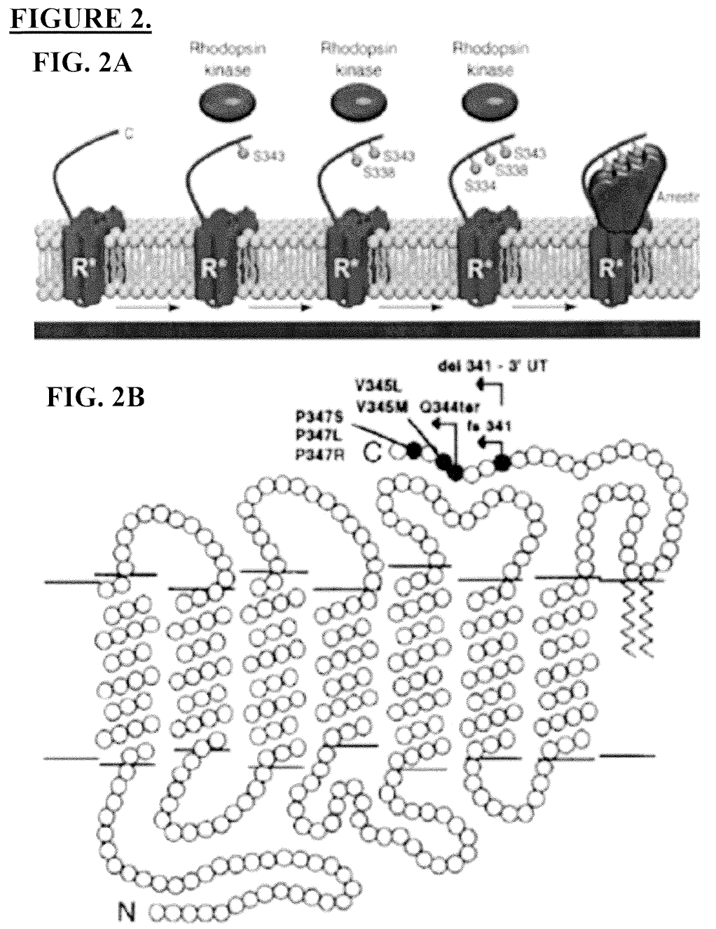

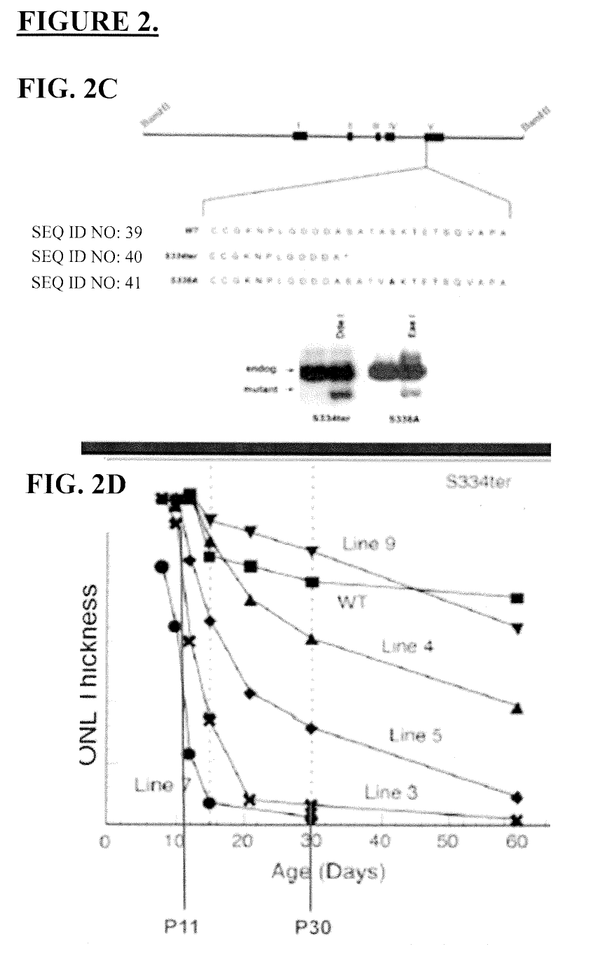

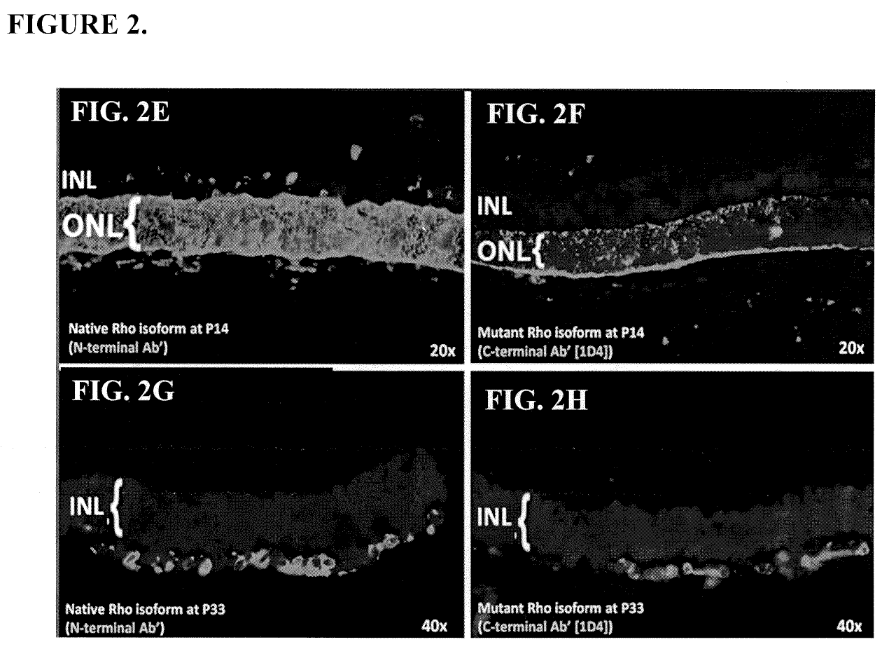

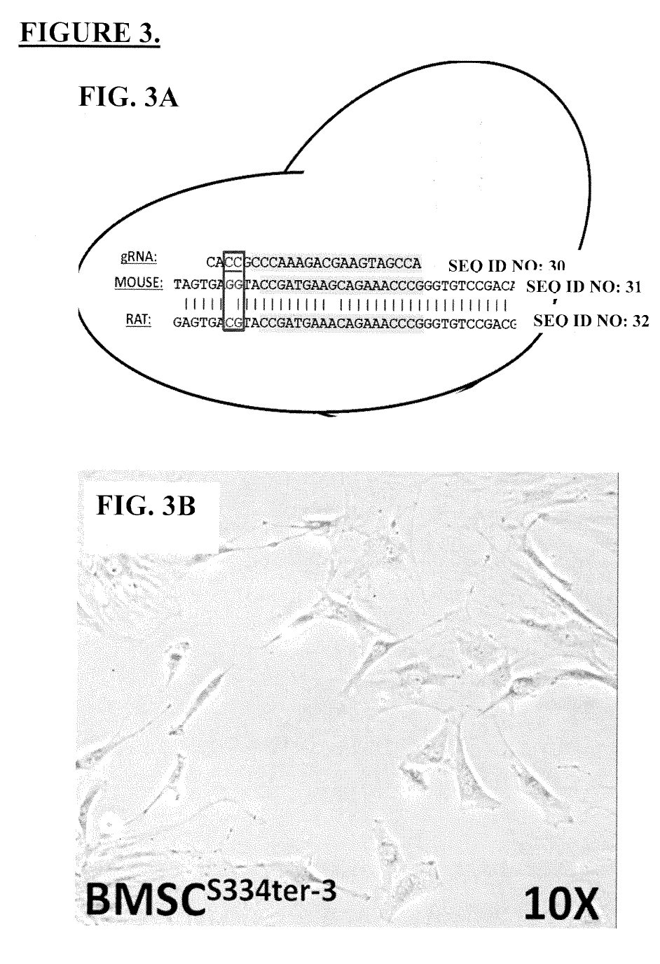

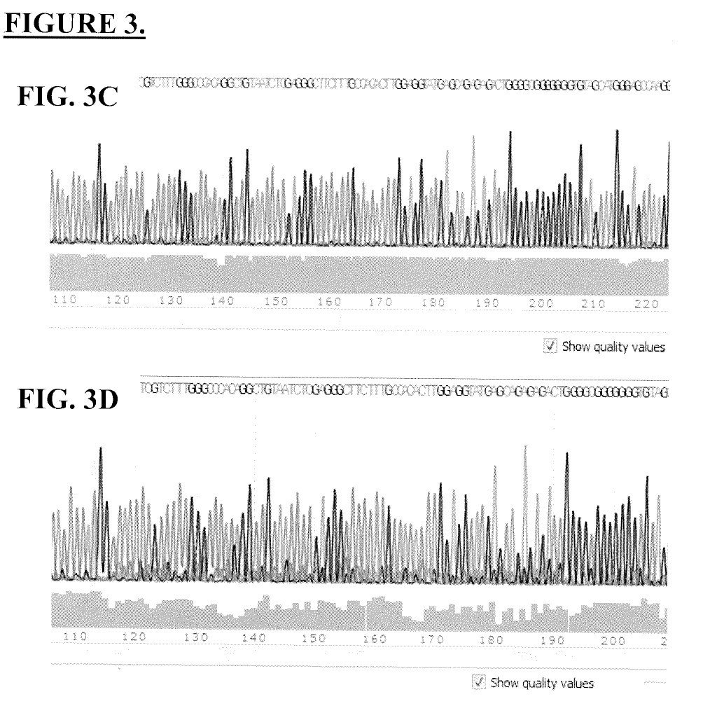

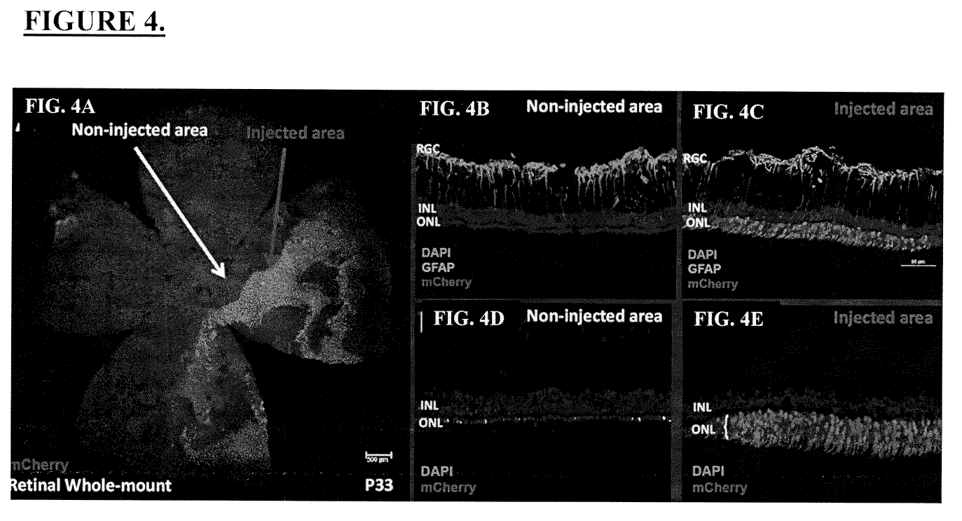

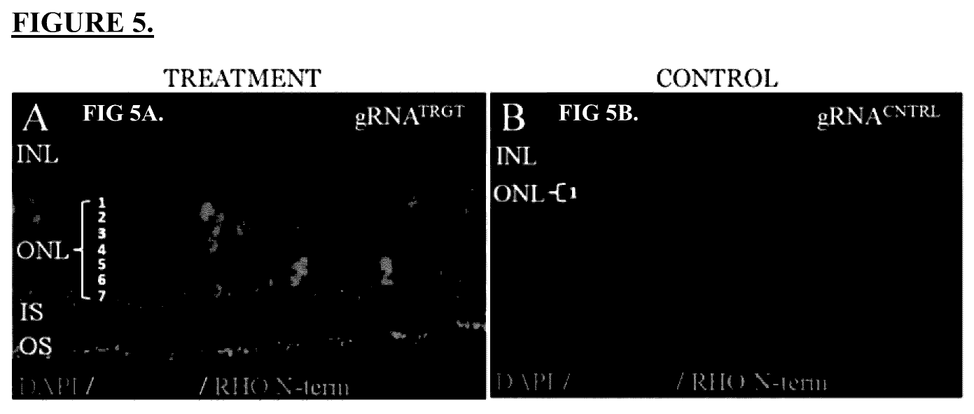

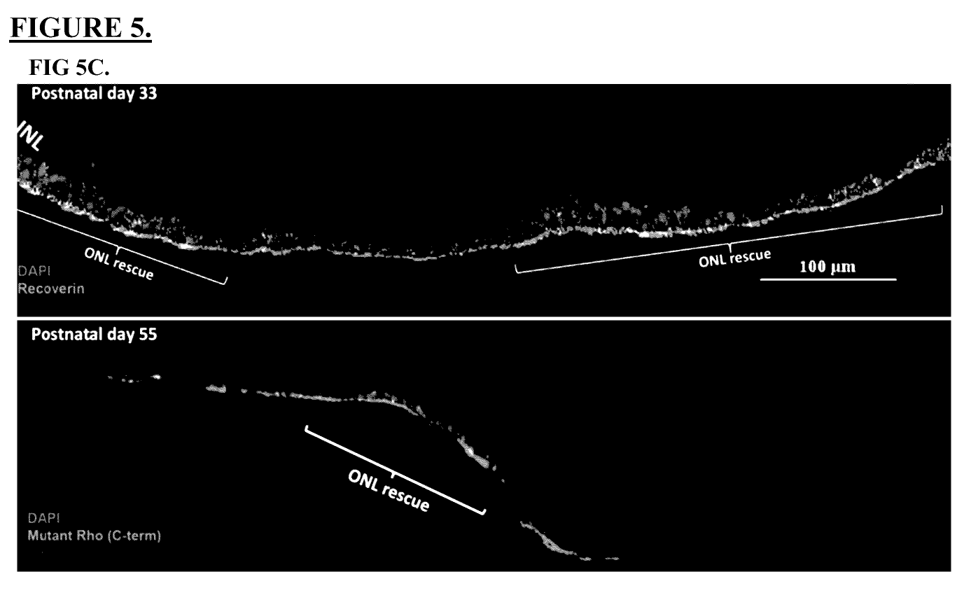

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Indeed, the present invention is in no way limited to the methods described herein. For purposes of the present invention, the following terms are defined below.

“Administering” and/or “administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

“Modulation” or “modulates” or “modulating” as used herein refers to upregulation (i.e., activation or stimulation), down regulation (i.e., inhibition or suppression) of a response or the two in combination or apart.

“Pharmaceutically acceptable carriers” as used herein refer to conventional pharmaceutically acceptable carriers useful in this invention.

“Promote” and/or “promoting” as used herein refer to an augmentation in a particular behavior of a cell or organism.

“Subject” as used herein includes all animals, including mammals and other animals, including, but not limited to, companion animals, farm animals and zoo animals. The term “animal” can include any living multi-cellular vertebrate organisms, a category that includes, for example, a mammal, a bird, a simian, a dog, a cat, a horse, a cow, a rodent, and the like. Likewise, the term “mammal” includes both human and non-human mammals.

“Therapeutically effective amount” as used herein refers to the quantity of a specified composition, or active agent in the composition, sufficient to achieve a desired effect in a subject being treated. A therapeutically effective amount may vary depending upon a variety of factors, including but not limited to the physiological condition of the subject (including age, sex, disease type and stage, general physical condition, responsiveness to a given dosage, desired clinical effect) and the route of administration. One skilled in the clinical and pharmacological arts will be able to determine a therapeutically effective amount through routine experimentation.

“Treat,” “treating” and “treatment” as used herein refer to both therapeutic treatment and prophylactic or preventative measures, wherein the object is to prevent or slow down (lessen) the targeted condition, disease or disorder (collectively “ailment”) even if the treatment is ultimately unsuccessful. Those in need of treatment may include those already with the ailment as well as those prone to have the ailment or those in whom the ailment is to be prevented.

As described, retinitis pigmentosa (RP) is a heterogenous group of inherited retinal degenerations resulting from multiple possible gene mutations that cause initial loss of night vision followed by progressive loss of visual acuity and progressive blindness. Heterogeneity in RP poses a significant challenge to development of therapeutic strategies and precludes understanding of the underlying mechanisms and pathophysiology of the disease. Nevertheless, mutations in rhodopsin (RHO), a critical molecule in the visual cascade, accounts for a significant number of those afflicted. Current mutation-focused approaches for treating RP have delivered promising results, but with significant limitations. For example, one type of gene supplementation therapy targeting RP at the level of the gene mutation. This includes RPE65 gene therapy for RPE65-related early onset retinal dystrophy, a particulate form of RP. Results indicated successful rescue of visual function and improved pupillary light reflex in pediatric patients. Replacement of the REP1 gene for choroideremia similarly found improved visual acuity and retinal sensitivity. But for applications such as RPE65, functional rescue effect was coupled with unmitigated structural and cellular degeneration. Such results suggest current mutation focused approaches provide only a limited window of efficacy for gene therapy, limited by treatment magnitude and/or timeliness, where insufficient or late addition of the normal gene copy to retinal cells will fail to halt or modify the disease course.

As a result, gene editing tools using bacterial Clustered Regularly Interspaced Short Palindromic Repeats (CRISPR) and CRISPR-associated protein (cas) system provides a totally new therapeutic avenue, wherein gene editing of the dominant negative mutation form of the disease could stymie progressive vision loss in RP. In particular, dominant negative form of RP requires that only one mutated copy of the gene suffice to initiate RP disease pathogenesis, whether by reduction in the level of wild-type protein (haploinsufficiency), by a gain of a deleterious function (dominant negative effect), or a combination of the two. Dominant mutations may also lead to disease by causing the buildup of toxic proteins. Simple gene-replacement therapy is often insufficient to overcome the presence and expression of the mutant allele. Because there can be many disease-causing dominant mutations in a single gene, targeted gene elimination or repair for each separate mutation is problematic or infeasible using existing approaches. An alternative approach would be to block production of toxic actors, promote cell survival and clearance of toxic buildup, preserve affected retinal cells, thereby slowing the course of degeneration.

More specifically, a common mutation associated with autosomal dominant RP includes mutations in rhodopsin (RHO) or the peripherin (RDS) gene. As the first mutation described for RP, disruption in this visual pigment rhodopsin (RHO) gene in rod-photoreceptor cells leads to the impaired vision associated with RP. This impartment in RHO function is highly deleterious by disabling a critical first step in phototransduction. Consisting of a protein moiety, an opsin, and a nonprotein moiety chromophore 11-cis-retinal, opsin is a hepatrimeric G-protein-coupled receptor (GPCR), localized predominantly in the disk membranes of rod outer segments, whereupon isomerization of 11-cis-retinal to all-trans-retinal upon absorption of a photon induces changes in opsin structure. This change activates the G protein transducin, thus initiating the biochemical cascade known as phototransduction. More than 120 mutations located in all three domains of RHO—intradiskal, transmembrane, and cytoplasmic—are associated with RP, which is perhaps unsurprising given the intricate conformational steps associated with the opsin protein and its role in phototransduction. Importantly, almost all mutations in RHO lead to the production of aberrant protein, accounting for the dominant negative function of RHO in RP.

For example, a rhodopsin mutation encoding a proline-to-histidine substitution at position 23 (P23H) results in P23H rhodopsin mutants that are retained in the endoplasmic reticulum and are unable to associate with 11-cis-retinal. Unable to be degraded by the ubiquitin-proteasome system, large quantities of unfolded, mutant protein accumulate as ubiquitinated P23H in the cytoplasm. Similar to other dominant inherited neurodegenerative diseases, such as Parkinson's and amyotrophic lateral sclerosis (ALS), the formation of intracellular protein aggregates of abberant protein is associated with cellular degeneration. This toxic gain of function from misfolded RHO induces degeneration of photoreceptors, so as to lead to dominant disease penetrance, whereby mutations of only one allele can lead to visual impairment. Another similar mutation is peripherin, a transmembrane glycoprotein that, along with an associated protein, retinal outer-segment membrane, is localized to the rim region of outer-segment disks in rods and cones. RDS mutations in humans display an autosomal dominant pattern of inheritance with over 90 human mutations in RDS identified. A common feature of these disorders is the loss of macular (central retinal) photoreceptors, a phenotype also seen throughout the RDS−/− mouse retina.

Gene replacement strategies in clinical trials for retinal degenerative diseases were designed to compensate for the biallelic inheritance of recessive, loss-of-function mutations. This approach, however, is inapplicable to adRP in which disease penetrance is conferred by a monoallelic, gain-of function mutation. Twenty-four genes have been implicated in adRP etiology, with Rho variations constituting the highest proportion of RP cases. Studies in transgenic animals bearing dominant Rho mutations showed that disease severity can be mitigated by silencing the mutant RNA transcript, or via transcriptional suppression using an allele-independent approach to target both mutant and wild-type genes. The caveat to these approaches, however, is the requisite supplementation with the wild-type Rho (RhoWT) transcript.

Allele-specific genomic ablation using CRISPR/Cas9 may present a simplified therapeutic strategy in which retinal function is restored by the remaining RhoWT allele in adRP patients. Though two RhoWT alleles will remain following transgene ablation in the model used here, patient hemizygosity does not manifest in haploinsufficiency as RhoWT expression between 50 and 200% is clinically asymptomatic. Moreover, as little as 10% of total Rho expression from a control transgene was shown to be sufficient to reconstitute the WT phenotype in Rho knockout mice. Accordingly, adRP may be amenable to allele specific ablation therapy without requiring exogenous RhoWT supplementation.

Transgenic S334ter rats that possess the mouse genomic fragment containing Rho_{S334} show phenotypic similarity to human class-I RHO mistrafficking mutations; exhibiting continual PR loss and commensurate vision decline. The S334ter mutation generates a serine substitution at amino acid position 334/338 for a stop codon resulting in early termination (“ter”), and RHO truncation by 15 Cterminal residues. The resultant peptide (RHOS334) lacks three serines required for photoreceptor (PR) deactivation following light stimulation, and part of the signal sequence required for RHO trafficking to photoreceptor outer segments. The morphological development of POS is critical to support phototransduction, and is absent in line-3 S334ter rats (S334ter-3). As RHO constitutes approximately 85% of the total protein content in PRs, its retention in processing organelles at the cell body instigates PR toxicity, and is compounded by constitutive PR activity, resulting in apoptosis.

The Inventors hypothesized that the selective ablation of RhoS334 in vivo would eliminate the RHOS334 production and toxicity, and allow native RhoWT to restore the phenotype to that of the non-dystrophic retina. The Inventors used CRISPR/Cas9 to selectively disrupt RhoS334 by utilizing the requirement of Cas9 activity on the recognition of a protospacer adjacent motif (PAM) present in RhoS334, which diverges from the RhoWT sequence by one nucleotide (5′-TGG-3′ versus 5′-TGC-3′, respectively). Here The Inventors show that this single base pair difference facilitated discrimination between Rho alleles during Cas9 cleavage, which permitted the unabated function of RhoWT to prevent RP pathology and loss of visual acuity.

### Genomic Editing.

In view of the heterogeneous genetic underpinnings governing RP disease initiation and progression, genome engineering is versatile and powerful tool to correct genetic mutations. Site-specific chromosomal integration can target desired nucleotide changes, including introducing therapeutic gene cassettes in safe landing sites within chromosomes, disrupting the coding or non-coding regions of specific alleles and correcting the genetic mutations to reverse the disease phenotype. Conventional technologies such as Zinc Finger Nuclease (ZFNs) and Transcription Activator-Like Effector Nucleases (TALENs) have provided a significant groundwork of proof-of-concept studies for genome editing and therapy. Yet, the most recent advances in Clustered Regularly Interspaced Short Palindromic Repeats (CRISPR) and CRISPR-associated endonuclease protein (cas) system extend this versatility and convenience by reducing the number of steps required for designing targeting of a particular mutation.

Briefly, genome editing using tools such as ZFNs can be based on the introduction of a site-specific DNA double stranded break (DSB) into the locus of interest. Key to this process is the cellular repair mechanism for efficiently repairing DSBs via the homology-directed repair (HDR), or non-homologous end joining (NHEJ) pathways. The mechanisms of these DNA repair pathways can generate defined genetic outcomes. More specifically, genome editing using ZFNs can be based on the introduction of a site-specific DNA DSB into the locus of interest. Thereafter, NHEJ repair, can rapidly and efficiently ligate two broken ends, providing opportunity for the gain or loss of genetic information. This feature can be exploited to introduce small insertions and/or deletions at the site of the break, thereby allowing disruption of a target gene. If, for example, a disease results from toxic protein buildup, instruction of a nonsense or missense sequence effectively eliminates aberrant protein to correct human disorders caused by inherited gene defects. Alternatively, if a specifically-designed homologous donor DNA is provided in combination with the ZFNs, this template can result in gene correction or insertion, as repair of the DSB can include a few nucleotides changed at the endogenous site or the addition of a new gene at the break site.

While pioneering much of what is known about genomic editing process, significant challenges exist with conventional technologies such as ZFNs, and TALENs. These early generation nucleases, ZFNs and TALENs are artificial fusion proteins composed of an engineered DNA binding domain fused to a non-specific nuclease cleavage domain from the FokI restriction enzyme. Zinc finger and transcription activator-like effector repeat domains with customized specificities can be joined to bind to extended DNA sequences. While adaptation of ZFNs and TALENs by modifying the DNA-binding specificities provide a significant level of targeting control, individual zinc finger domains provide some heterogeneity requiring some context-dependence for DNA binding. TALE repeat domains appear less susceptible to these context-dependent effects and can be modularly assembled to recognize virtually any DNA sequence via a simple one-to-one code between individual repeats and the four possible DNA nucleotides, but assembly of DNAs encoding large numbers of highly conserved TALE repeats can require the use of non-standard molecular biology cloning methods.

Whereas both ZFNs and TALENs involving use protein-DNA interactions for targeting, bacterial CRISPR-Cas system is unique and flexible due to utilization of RNA as the moiety that targets the nuclease to a desired DNA sequence. In contrast to ZFN and TALEN platforms, CRISPR-CAS uses simple Watson-Crick base pairing rules between an engineered RNA and the target DNA site. Generally, two components form the core of a CRISPR nuclease system, a Cas nuclease (e.g., cas9) and a guide RNA (grNA), the gRNA derived from a fusion of CRISPR-derived RNAs (“crRNA”) and trans-acting antisense RNA (“tracRNA”). In the most well-studied example, the single gRNA complexes with a cas protein (e.g., cas9) to mediate cleavage of target DNA sites that are complementary to the first (5′) 20 nts of the gRNA and that lie next to a protospacer adjacent motif (“PAM”) sequence (canonical form of 5′-NGG for Streptococcus pyogenes cas9, but also alternate 5′-NAG exist). Thus, with this system, Cas9 nuclease activity can be directed to any DNA sequence of the form N20-NGG simply by altering the first 20 nts of the gRNA to correspond to the target DNA sequence. It is notes that Type II CRISPR systems from other species of bacteria recognize alternative PAM sequences and that utilize different crRNA and tracrRNA sequences could also be used to perform targeted genome editing.

The Cas9-induced DSBs have been used to introduce NHEJ-mediated indel mutations as well as to stimulate HDR with both double-stranded plasmid DNA and single-stranded oligonucleotide donor templates. The capability to introduce DSBs at multiple sites in parallel using the Cas9 system is a unique advantage of this platform relative to ZFNs, or TALENs. For example, expression of Cas9 and multiple gRNAs has been used to induce small and large deletions or inversions between the DSBs, to simultaneously introduce parallel genetic editing mutations altering different genes in rats, mouse ES cell clones, and zebrafish. Together, these advances in CRISPR/cas-mediated gene editing technology can accelerated the pace of gene-function relationship discovery, and a focused approach for developing personalized therapeutics.

Despite these advances, the extent of clinical applicability to patients through direct in vivo genome modification is not yet clear. The elegance of the CRISPR/Cas system as allowing for tailoring to target the patient's particular mutation, combined with a delivery system via adeno-associated virus (“AAV”), or also via adenovirus, vectors as optimal vehicles for genome editing machinery can deliver components directly to the organ or cells of interest. There have been limited reports that, for example, systemic injection of an AAV vector carrying a zinc-finger nuclease and donor template construct was able to correct mutant transgenic clotting Factor IX in mice and reconstitute low but clinically detectable levels of circulating protein. In this regard, an AAV-CRISPR system could be delivered directly and locally to treat the diseased retina with some notable advantages. It is noted that S. Pyogenes Cas9 transcript used in the described study may be too large to be packaged into AAV particles, which necessitated in vivo electroporation of plasmid DNA at postnatal day 0. However, the smaller transcript of the S. aureus Cas9 allows packaging into AAV particles for translational in vivo delivery at postnatal day 15. Importantly, the PAM of SaCas9 is NNGRRT, different from that of SpCas9 (NGG). In this aspects, one can rely on SaCas9 constructs to be packaged into AAV particles in which the RhoS334ter transgene is targeted in S334ter-3 rats.

This includes the ability to address dominant mutations via the same gene correction mechanism used for recessive mutations, compactly deliver targeted genomic editing machinery with a limited footprint capable of being delivered via viral vectors, as constant and agnostic to the size of the target gene and maintain the endogenous gene expression stoichiometry. These and other advantages of CRISPR/Cas editing give it a wide range of possible clinical applications.

Toward these ends, the Inventors demonstrate proof-of-concept of prophylactic prevention and reversal of retinal defects in an animal model of retinitis pigmentosa (RP) through targeted gene modification using CRISPR. Specifically, the Inventors demonstrate that CRISPR/Cas9 technology can be used in living animals to prophylactically prevent a genetic disease from manifesting, based on targeting and disruption of the specific the mutated RHO gene responsible for causing the disease in the retinal cells of a transgenic rat model. Non-mutated copy of the gene (2 copies for each gene) is not targeted for destruction and functions normally without interference from the presence of the mutated, dominant gene, which prevented retinal degeneration. While conditions such as blindness from recessive gene mutations can also be corrected by supplying the missing gene using the aforementioned method and compositions (such as replacement of the RPE65 gene), a key advance here is addressing those subjects with dominant genetic mutations more difficult to treat due to lack of a gene product, or a toxic gene product. The existence of a mutated gene product, inhibiting or diluting, proper function of non-mutated gene copy provides the dominant negative effect. To date, such disease conditions have been attempted using techniques such as ribozyme therapy in animals to treat dominant RP, but none have involved the use of CRISPR as described. For dominant RP one of two gene copies is mutated, a proposed therapy intends to destroy both copies and subsequently deliver new copies that are not able to be destroyed by the method used in phase-1 (targeted genetic ablation) of the therapy. The Inventors' technology effectively deletes the mutated copy allowing the normal function of the wild-type copy to prevent cell and vision loss.

Reliable genome editing via Clustered Regularly Interspaced Short Palindromic Repeat (CRISPR)/Cas9 may provide a means to correct inherited diseases in patients. As proof of principle, The Inventors show that CRISPR/Cas9 can be used in vivo to selectively ablate the rhodopsin gene carrying the dominant S334ter mutation (Rho_{S334}) in rats that model severe autosomal dominant Retinitis Pigmentosa (adRP). A single subretinal injection of guide RNA (gRNA)/Cas9 plasmid in combination with electroporation generated allele-specific disruption of Rho_{S334}, which prevented retinal degeneration and improved visual function.

Described herein is a method of treatment including providing a quantity of one or more therapeutic vectors and administering the one or more therapeutic vectors to a mammal afflicted with a disease and/or condition, wherein in vivo expression of the one or more therapeutic vector is capable of treating the mammal for the disease and/or condition. In other embodiments, the one or more therapeutic vectors, each encode at least one clustered regularly interspaced short palindromic (CRISPR) protein and one or more guide RNAs (gRNAs). In other embodiments, the CRISPR protein is a Streptococcus pyogenes-derived cas protein. In other embodiments, the CRISPR protein is not a Streptococcus pyogenes-derived cas protein. In other embodiments, the CRISPR protein is cas9. In other embodiments, the one or more gRNAs include a sequence capable of binding to a protospacer adjacent motif (“PAM”). In other embodiments, the PAM includes the sequence NGG. In other embodiments, the PAM includes the sequence NAG. In other embodiments, the PAM is NNGRRT, such as that used for S. Pyogenes. In other embodiments, the PAM is NGG, such as that used for S. Aureus. In other embodiments, the gRNA comprise a CRISPR-derived RNAs (“crRNA”) and trans-acting antisense RNA (“tracRNA”). In various embodiments, the gRNA is 10, 20, 30, or 40 or more nucleotides in length. In various embodiments, about 15, 16, 17, 18, 19, 20, 21, 22, 23, 24, 25 or more nucleotides are cognate to a gene of interest. As used herein and throughout the application, cognate includes sequences that are exactly complementary to each other, or substantially complementary to each other (e.g., 80, 85, 90, 95% complementary based on base pair sequence identity). In various embodiments, about 20 nucleotides are cognate to a genetic loci of interest. For example, this includes gRNA designs that hybridize to a target sequence with N₂₀NGG. In other embodiments, the disease and/or condition includes a single gene mutation. Various example include cystic fibrosis, sickle cell disease, Fragile X syndrome, muscular dystrophy. In other embodiments, the disease and/or condition includes a dominant mutation. In various embodiments, dominance is characterized by toxic gain of function, loss of function and/or haploinsufficiency. Various examples include amyotrophic lateral sclerosis (ALS), Huntington's disease, neurofibromatosis type 1 and 2, Marfan syndrome, nonpolyposis colorectal cancer, Von Willebrand disease, among many others. In other embodiments, the disease and/or condition including a dominant mutation is retinitis pigmentosa (RP). In other embodiments, the RP includes a mutation in rhodopsin (RHO) or peripherin (RDS). In other embodiments, the RP includes a mutation in RPGR, PRPH, RDH12, CRX, ROM1, RP1, PRPF31, PRPF3, PRPF8, IMPDH1, NRL, CA4, FSCN2, GUCA1B, RP9, and SEMA4A. In other embodiments, the mammal includes a human. In other embodiments, the therapeutic vector includes an adenovirus, adeno-associated virus, or lentivirus. In other embodiments, administering the one or more therapeutic vectors includes injection, inhalation, or infusion. In various embodiments, methods of administration to the subject will depend on the delivery mechanism. For example, the aforementioned editing constructs may be delivered as nucleotides using vectors, or as assembled protein/peptides, such as modular peptides, ocular delivery peptides, that could be introduced via injection or liposomes. In other embodiments, administering the one or more therapeutic vectors includes subretinal injection. In other embodiments, treating the mammal for the disease and/or condition includes in vivo generation of a double stranded break (DSB) in a population of cells in the mammal. In some embodiments, a single stranded break occurs (SSB). In other embodiments, treating the disease and/or conditions includes in vivo homologous recombination (HR) of a DSB. In other embodiments, HR includes non-homologous end joining (NHEJ) introducing missense or nonsense of a protein expressed at the locus. In other embodiments, HR includes homology directed repair (HDR) introducing co-administered template DNA. In other embodiments, the co-administered template DNA is cognate to a wild-type genetic sequence. In other embodiments, the disease and/or condition includes a recessive mutation. In some embodiments, the HR results in an alteration that is an indel. In some embodiments, the HR results in an alteration causing reduced expression of the target polynucleotide sequence. In some embodiments, the HR results in an alteration that abrogates expression of a protein and/or polypeptide from the target polynucleotide sequences. In some embodiments, the alteration results in a knock out of the target polynucleotide sequence. In some embodiments, the HR results in an alteration that adjusts the target polynucleotide sequence from an undesired sequence to a desired sequence. In some embodiments, the alteration is a homozygous alteration. In some embodiments, each alteration is a homozygous alteration. In various embodiments, a quantity of stem cells, or cells differentiated from stem cells, are administered simultaneously or sequentially. Such cells can include autologous cells, including cells with alteration of a target polynucleotide sequence in the cell or cells via the described methods and compositions.

Further described is an in vivo method of genomic editing including providing a quantity of one or more vectors each encoding at least one clustered regularly interspaced short palindromic (CRISPR) protein and one or more guide RNAs (gRNAs) and administering the one or more vectors to a mammal, wherein in vivo expression of the one or more vectors includes binding of the CRISPR protein to a locus cognate to the gRNA and in vivo generation of a double stranded break (DSB) in a population of cells in the mammal, wherein in vivo homologous recombination (HR) of the DSB results in editing of the genome of a population of cells in the mammal. In other embodiments, the CRISPR protein is cas9 and the one or more gRNAs include a sequence capable of binding to a protospacer adjacent motif (“PAM”). In other embodiments, HR includes non-homologous end joining (NHEJ) introducing missense or nonsense of a protein expressed at the locus. In other embodiments, HR includes homology directed repair (HDR) introducing template DNA co-administered in step (b). In other embodiments, the disease and/or condition includes a dominant mutation. In other embodiments, the disease and/or condition includes a recessive mutation. In other embodiments, the vector includes an adenovirus or lentivirus. In other embodiments, the disease and/or condition including a dominant mutation includes retinitis pigmentosa (RP) including a mutation in rhodopsin (RHO) or peripherin (RDS), the mammal includes a human, and administering the one or more therapeutic vectors includes subretinal injection. In other embodiments, administering the one or more vectors includes injection, inhalation, or infusion. In various embodiments, methods of administration to the subject will depend on the delivery mechanism. For example, the aforementioned editing constructs may be delivered as nucleotides using vectors, or as assembled protein/peptides, such as modular peptides, ocular delivery peptides, that could be introduced via injection or liposomes. In other embodiments, administering the one or more vectors includes subretinal injection. In other embodiments, in vivo method of genomic editing includes generation of a double stranded break (DSB) in a population of cells in the mammal. In some embodiments, a single stranded break occurs (SSB). In other embodiments, in vivo method of genomic editing includes homologous recombination (HR) of a DSB. In other embodiments, HR includes non-homologous end joining (NHEJ) introducing missense or nonsense of a protein expressed at the locus. In other embodiments, HR includes homology directed repair (HDR) introducing co-administered template DNA. In other embodiments, the co-administered template DNA is cognate to a wild-type genetic sequence. In other embodiments, the disease and/or condition includes a recessive mutation. In some embodiments, the HR results in an alteration that is an indel. In some embodiments, the HR results in an alteration causing reduced expression of the target polynucleotide sequence. In some embodiments, the HR results in an alteration that abrogates expression of a protein and/or polypeptide from the target polynucleotide sequences. In some embodiments, the alteration results in a knock out of the target polynucleotide sequence. In some embodiments, the HR results in an alteration that adjusts the target polynucleotide sequence from an undesired sequence to a desired sequence. In some embodiments, the alteration is a homozygous alteration. In some embodiments, each alteration is a homozygous alteration.

Further described is an in vitro method of genomic editing including providing a quantity of one or more vectors each encoding at least one clustered regularly interspaced short palindromic (CRISPR) protein and one or more guide RNAs (gRNAs) and administering the one or more vectors to a mammal, wherein in vitro expression of the one or more vectors includes binding of the CRISPR protein to a locus cognate to the gRNA and in vitro generation of a double stranded break (DSB) in a population of cells in the mammal, wherein in vitro homologous recombination (HR) of the DSB results in editing of the genome of a population of cells in the mammal. In other embodiments, the CRISPR protein is cas9 and the one or more gRNAs include a sequence capable of binding to a protospacer adjacent motif (“PAM”). In other embodiments, HR includes non-homologous end joining (NHEJ) introducing missense or nonsense of a protein expressed at the locus. In other embodiments, HR includes homology directed repair (HDR) introducing template DNA co-administered in step (b). In other embodiments, the disease and/or condition includes a dominant mutation. In other embodiments, the disease and/or condition includes a recessive mutation. In other embodiments, the vector includes an adenovirus or lentivirus. In other embodiments, the disease and/or condition including a dominant mutation includes retinitis pigmentosa (RP) including a mutation in rhodopsin (RHO) or peripherin (RDS), the mammal includes a human, and administering the one or more therapeutic vectors includes subretinal injection. In other embodiments, administering the one or more vectors includes injection, inhalation, or infusion. In various embodiments, methods of administration to the subject will depend on the delivery mechanism. For example, the aforementioned editing constructs may be delivered as nucleotides using vectors, or as assembled protein/peptides, such as modular peptides, ocular delivery peptides, that could be introduced via injection or liposomes. In other embodiments, administering the one or more vectors includes subretinal injection. In other embodiments, in vitro method of genomic editing includes generation of a double stranded break (DSB) in a population of cells in the mammal. In some embodiments, a single stranded break occurs (SSB). In other embodiments, in vitro method of genomic editing includes homologous recombination (HR) of a DSB. In other embodiments, HR includes non-homologous end joining (NHEJ) introducing missense or nonsense of a protein expressed at the locus. In other embodiments, HR includes homology directed repair (HDR) introducing co-administered template DNA. In other embodiments, the co-administered template DNA is cognate to a wild-type genetic sequence. In other embodiments, the disease and/or condition includes a recessive mutation. In some embodiments, the HR results in an alteration that is an indel. In some embodiments, the HR results in an alteration causing reduced expression of the target polynucleotide sequence. In some embodiments, the HR results in an alteration that abrogates expression of a protein and/or polypeptide from the target polynucleotide sequences. In some embodiments, the alteration results in a knock out of the target polynucleotide sequence. In some embodiments, the HR results in an alteration that adjusts the target polynucleotide sequence from an undesired sequence to a desired sequence. In some embodiments, the alteration is a homozygous alteration. In some embodiments, each alteration is a homozygous alteration.

Further described herein is a composition including a vector encoding a regularly interspaced short palindromic (CRISPR) protein and one or more guide RNAs (gRNAs), wherein the one or more gRNAs include a sequence capable of binding to a protospacer adjacent motif (“PAM”). In other embodiments, the vector encodes at least one clustered regularly interspaced CRISPR protein and one or more gRNAs. In other embodiments, the CRISPR protein is a Streptococcus pyogenes-derived cas protein. In other embodiments, the CRISPR protein is not a Streptococcus pyogenes-derived cas protein. In other embodiments, the CRISPR protein is cas9. In other embodiments, the one or more gRNAs include a sequence capable of binding to a PAM. In other embodiments, the PAM includes the sequence NGG. In other embodiments, the PAM includes the sequence NAG. In other embodiments, the PAM is NNGRRT, such as that used for S. Pyogenes. In other embodiments, the PAM is NGG, such as that used for S. Aureus. In other embodiments, the gRNA comprise a CRISPR-derived RNAs (“crRNA”) and trans-acting antisense RNA (“tracRNA”). In various embodiments, the gRNA is 10, 20, 30, or 40 or more nucleotides in length. In various embodiments, about 15, 16, 17, 18, 19, 20, 21, 22, 23, 24, 25 or more nucleotides are cognate to a gene of interest. In various embodiments, about 20 nucleotides are cognate to a genetic loci of interest. For example, this includes gRNA designs that hybridize to a target sequence with N₂₀NGG. In some embodiments, the CRISPR protein is cas9 and the gRNA is cognate to a locus encoding rhodopsin (RHO) or peripherin (RDS). In various embodiments, the composition is used in a method for altering a target polynucleotide sequence in a cell including contacting the polynucleotide sequence with a CRISPR protein (e.g., cas9) with at least one gRNA directing CRISPR to hybridize to a cognate sequence on a target polynucleotide sequence, wherein the target polynucleotide sequence is cleaved, and wherein the efficiency of alteration of cells that express CRISPR protein is from about 50% to about 80%. Further described herein is a quantity of cells produced using the described method.

### Example 1

### Animal Model

The S334ter-line-3 rat is a transgenic model of retinal degeneration developed to express a rhodopsin mutation similar to that found in human retinitis pigmentosa (RP) patients. The S334ter-line-3 rat possess a mouse rhodopsin (RHO) gene bearing a termination codon at residue 334, which results in a C-terminal truncated RHO protein lacking the last 15 amino acid residues that is not trafficked to the outer segments. Heterozygous rats of the S334ter line-3 exhibit fast degeneration and due to the truncated rhodopsin sequestration, and never develop rod photoreceptor outer segments. Retinal degeneration occurs in the mean outer nuclear layer (ONL), and superior hemisphere is slightly more degenerated than the inferior hemisphere. S334ter-line-3 model also exhibits the hallmarks of cellular remodeling caused by photoreceptor degeneration including abnormal processes of bipolar cells, lower density of biopolar cells, and glial reactivity.

### Example 2

### CRISPR/Cas9 Constructs, Generally

The CRISPR associated protein cas9 is utilized to induce double-stranded DNA break at the mutant RHO in S334ter-line-3, with the DNA break repaired so as to prevent expression of the aberrant mutant rhodopsin by missense or nonsense mutation, thereby preventing toxic buildup of abberant protein normally causing cellular retinal degeneration as repaired by nonhomologous end-joining (NHEJ).

Utilizing this mechanism, CRISPR is especially promising for targeting gain-of-function mutations in which silencing of the mutated allele is sufficient to preserve the cell. Using Cas9, the sequence of the gene could be disrupted in a way that would prevent translation of that allele. Alternatively, homologous directed repair (HDR) could be used to incorporate a template sequence to correct a genetic mutation, such as normal wild-type RHO. In addition to targeted disruption (ablation) of a dominant allele, and targeted insertion by HDR, one could target disruption of recessive mutant alleles to correct frameshift mutations by restoring the open reading frames in endogenous genes bearing recessive mutations.

### Example 3

### CRISPR/Cas9 Constructs, Specifically

The design for the gRNA is to knockout the mutation type of rhodopsin gene-Rho S334 and maintains the integrity of the wild type rat RHO gene. Since CRISPR/Cas9 mediated high efficiency genome editing relies on the protospacer-adjacent motif (PAM) sequence NGG, a specific gRNA was designed. The gRNA cooperation with Cas9 has high efficiency of cleavage only in mouse RHO S334 mutation gene, not in rat wild type RHO (which is lack of PAM sequence). In addition, the gRNA sequence also contains one base mismatch compared with rat wild type Rho gene locus, which further ensured the specificity of targeting mutation gene. The gRNA Cas9 targeting site is on the first exon of RHO S334 gene. Upon cleavage by Cas9, the Rho S334 locus typically undergoes NHEJ or HR for repairing DNA damage. NHEJ can be harnessed to mediate gene knockouts, as indel occurring within a coding exon can lead to frameshift mutation and premature stop codon.

### Example 4

### Methods

Heterozygous S334ter-3 rats received unilateral subretinal injection of CRISPR/Cas9/guide-RNA constructs that were designed to target the mutant rhodopsin gene of S334ter-3 rats. Rhodopsin and scrambled guide-RNA constructs contained the mCherry reporter for later detection of transfected photoreceptors by fluorescence microscopy or for isolation by flow cytometry.

Retinas were removed and evaluated for photoreceptor preservation between three and 72 days following treatment. Dissociated S334ter-3 retinal cells were sorted on the basis of mCherry reporter expression for subsequent DNA and protein analyses. Immunohistochemical evaluation of photoreceptor rescue was performed using monoclonal antibodies that exclusively recognize the native rhodopsin isoform.

### Example 5

### Results

Correct guide-RNA targeting and Cas9 cleavage were confirmed by genomic DNA sequencing of S334ter-3 rat-derived bone marrow progenitor cells in vitro. Photoreceptors were preserved in rhodopsin-targeted CRISPR/Cas9/guide-RNA injected eyes, which had 4-6 rows of photoreceptors, compared with a single discontinuous layer of photoreceptors in control-injected and untreated eyes. Immunostaining for the functional rhodopsin isoform was observed in the outer segments of preserved photoreceptors, whereas outer segments in this model fail to develop. Visual function in treated S334ter-3 rats is currently being evaluated by electroretinography (ERG), optokinetic response (OKR) and luminescence threshold recording (LTR) from the superior colliculi (SC).

### Example 6

### Conclusions

This study demonstrated that CRISPR/Cas9 repaired the retinal defects associated with S334ter-3 rats by deleting the dominant negative function of mutant rhodopsin. Preservation of photoreceptors in injected animals suggests sufficient specificity of the designed CRISPR/cas9 constructs to target only the mutated allele. Long-term safety and efficacy are currently under investigation.

### Example 7

### Cas9/gRNA Vector Design

gRNAs were cloned into px330 vectors (Life Technologies, Carlsbad Calif.) via BbsI restriction enzyme site upstream of the scaffold gRNA sequence and the mCherry reporter (Addgene Inc., Cambridge Mass.) was cloned downstream of the Cas9 transcript, which was under constitutive expression by cytomegalovirus (CMV) promoter.

### Example 8

### Cas9 Cleavage Efficiency Determination

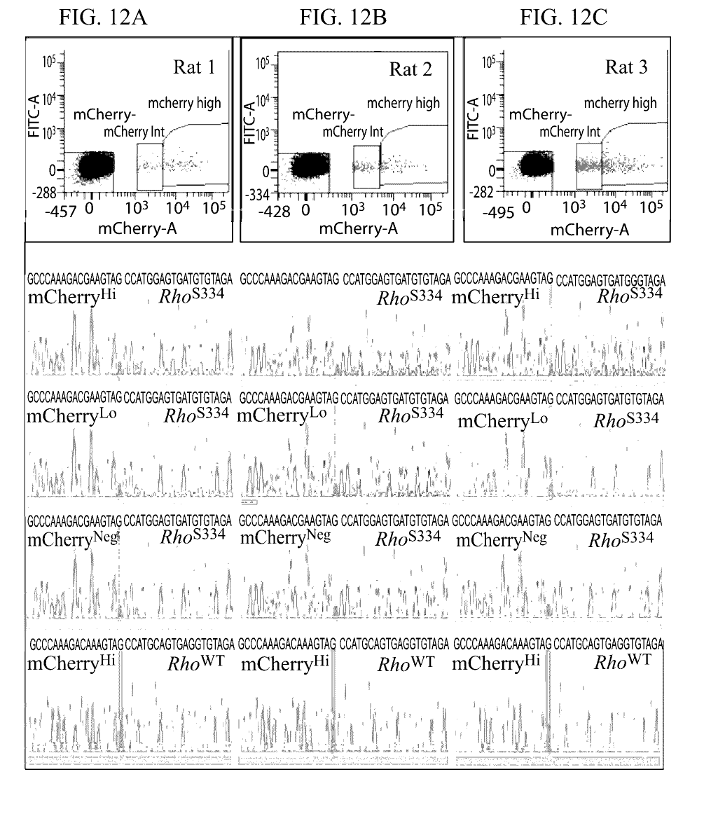

Rho_{S334}-selective disruption was confirmed in PRs that were FACS-isolated by mCherry_{Hi} and intermediate/low mCherry_{Lo} expression, and was confirmed absent in mCherry negative (mCherry_{Neg}) PRs from three animals (FIG. 12). Cleavage efficiency was calculated from the indel frequency among bacterial clones transfected with DNA from mCherry-isolated cells. Genomic DNA was extracted from mCherry+ PRs or MSCs and subjected to Polymerase Chain Reaction (PCR) amplification using primers that flanked the predicted Cas9 cut site regions. Amplicons from on- and off-target cleavage sites were ligated into plasmids (T-vector Cloning Kit; Life Technologies) and transfected into high-efficiency NEB-alpha E. coli (New England BioLabs, Ipswich Mass.) for DNA Sanger sequencing analysis of 21-42 clones from which the ratios of genomic disruptions were used to quantify cleavage efficiency (Table 2).

### Example 9

### Animal Procedures

Injection methods used were slightly modified from a previous published protocol. S334ter-3 P0 rats were anesthetized on ice for 5 minutes, and 1 μl of plasmid DNA (6-7.4 μg/μl) was diluted 0.1× (v/v) with Fast-Green DNA dye and subretinally injected by floating needle into S334ter-3 rats. Subsequent electroporation of plasmid DNA consisted of 5 pulses at 115 mV with 50 ms duration and 950 ms intervals, using a longitudinal sweeping motion with 7 mm Platinum Tweezertrodes lubricated with conductance enhancing SignaGel (ECM 830 System, Harvard Apparatus, Holliston Mass.). Positive charge was generated over the injected eye. Pups were allowed to recover on a heating pad.

### Example 10

### Cell Sources and Cell Processing

Eyes were surgically removed and kept in 4° C. PBS (pH 7.4) for approximately 30 minutes. Retinas were dissected and subjected to single-cell dissociation by incubating for 20 minutes at 37° C. in enzymatic digestion solution consisting of Ca₂₊/mg₂₊-free PBS, 20 U/ml papain, and 0.5 mM L-cysteine (Worthington Biochemical Corp., Lakewood N.J.). mCherry+ retinal cells were sorted (FACSAria III, BD Biosciences, Franklin Lakes N.J.) into 1.7 ml DNase-free tubes (Eppendorf, Hamburg Germany) containing 4° C. PBS, and subjected to genomic DNA extraction (Purelink Genomic DNA Mini Kit, Life Technologies). MSC_{SS334} were derived from adult rats as previously described. Briefly, the femurs of 6-8 week old S334ter-3 rats were flushed with DMEM (Life Technologies), gently triturated in 5 ml syringe (BD Biosciences), passed through a 40 μm strainer, centrifuged at 600×g for 10 min, plated at 1,000 cells/cm₂ onto T75 tissue culture flasks (Corning, Corning N.Y.) in growth medium consisting of DMEM containing 10% FBS (Atlanta Biologicals, Lawrenceville Ga.), 100 U/ml penicillin, and 100 μg/ml streptomycin sulfate (Life Technologies), and cultured in a humidified incubator at 37° and 5% CO₂. Non-adherent cells were removed by media change after 24 hrs.

### Example 11

### Immunofluorescent Staining and Confocal Microscopy

Eyes were enucleated and formalin-fixed (4% in PBS) (for 1 hour and embedded in OCT Compound (Sakura Finetek Inc., Torrance Calif.) after 30% sucrose infiltration. Select eyes were prepared for retinal whole-mount dissection to assess mCherry distribution prior to embedding. Frozen transverse 10 μm thick retinal sections were histologically stained (0.4% cresyl violet acetate, Sigma Aldrich, St. Lois Mo.) or immunolabeled with antibodies generated against: Cone-arrestin (rabbit polyclonal, 1:1,000; AB15282; Millipore, Billerica Mass.), PKC a (rabbit polyclonal, 1:5,000; P4334; Sigma), Synaptophysin (mouse clone SVP-38, 1:2,000; Millipore MAB368), Rhodopsin (C-terminal, clone 1D4, 1:100, Millipore MAB5356; or N-terminal, 1:100, clone RET-P1, Millipore MAB5316). Alexa-Fluor-488 (1:500; Life Technologies) was used to visualize sections along with nuclear counterstain (49,69-diamidino-2-phenylindole (DAPI; Vector Laboratories, Burlingame Calif.). Images were captured by confocal microscopy (Eclipse C1si; Nikon Instruments, Inc., Melville N.Y.) and morphology, mCherry distribution, PR nuclei counts analyzed by Image J software (NIH, Bethesda Md.).

### Example 12

### Visual Function Assessment

Animals were tested for spatial visual acuity by optomotry testing apparatus (CerebraMechanics, Lethbridge Canada) as previously described, in which four computer monitors are arranged in a square to project a 3D virtual space of a rotating cylinder lined with vertical sine wave grating. Unrestrained animals on a center platform tracked the projected image of rotating grating with reflexive head movements. The spatial frequency of the grating (cycles per degree) was centered on the rats' viewing position, and maximal acuity ascertained by increasing the grating frequency at psychophysics staircase progression until the tracking response was lost.

### Example 13

### Statistical Significance

Student's t-tests were performed using two-tailed distribution, and two-sample unequal variance (heteroscedastic) to compare OKR c/d values from treated vs. untreated eyes of individual animals, as well as between animals of treatment groups; gRNA_{TRGT} (N=5) and gRNA_{CNTRL} (N=4). Contralateral non-injected eyes served as untreated controls. Error bars indicate standard error mean (SEM). Statistical significance: *P≦0.05, **P≦0.01, ††P≦0.01, ***P≦0.001.

### Example 14

### Differential Distribution of RHO_{S334} Versus RHO_{WT} in S334ter-3 Rats

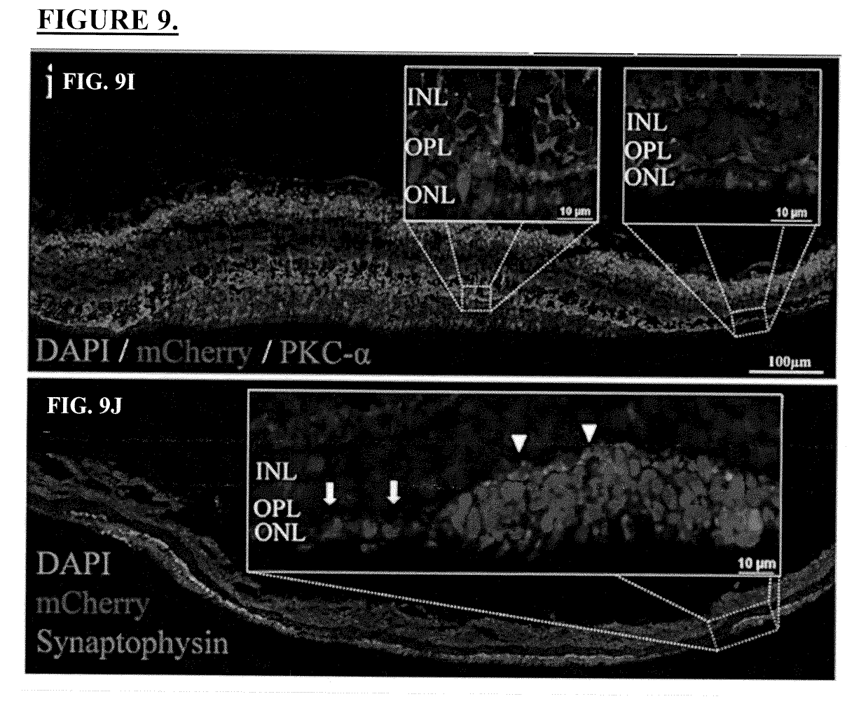

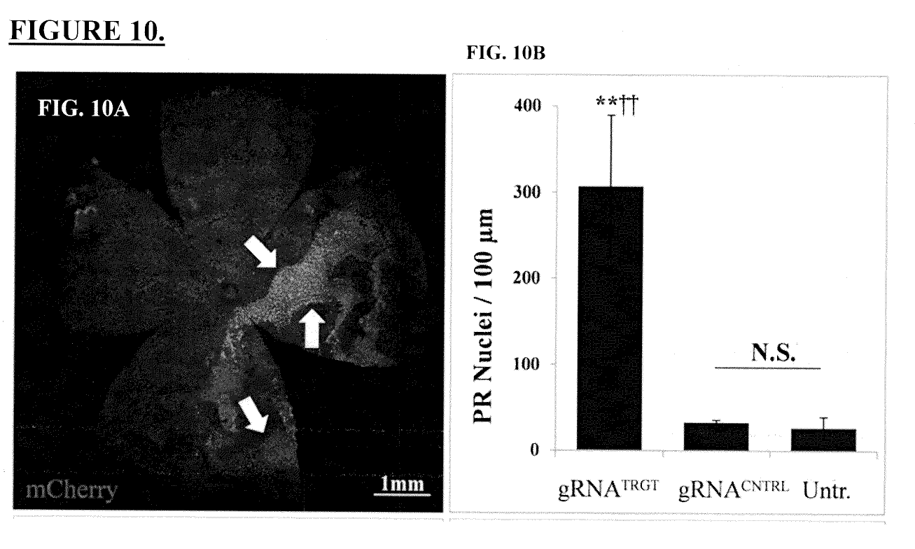

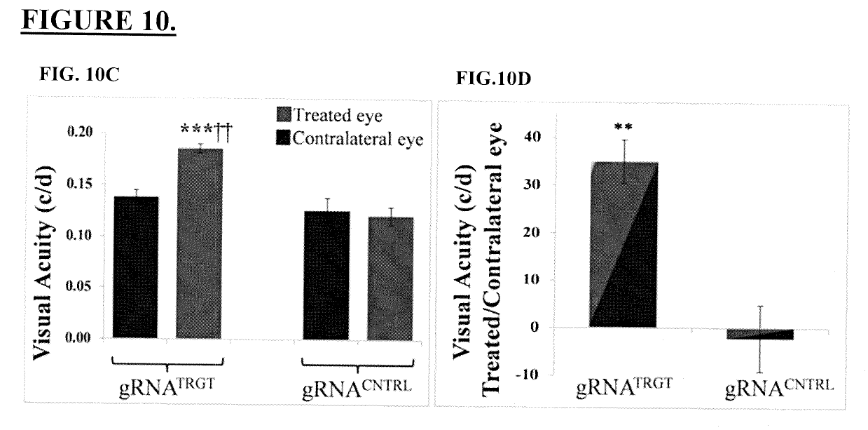

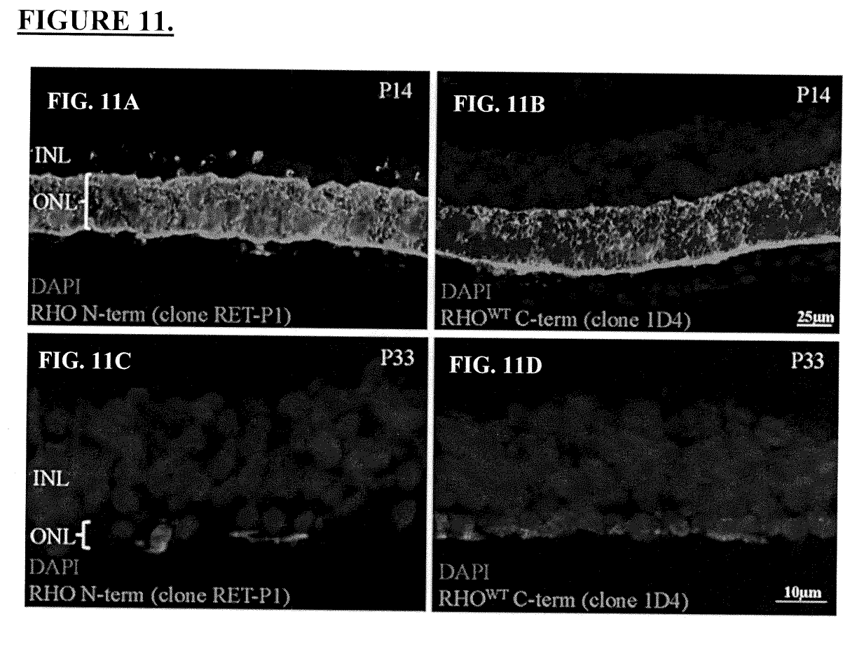

S334ter-3 rats are characterized by the rapid and progressive loss of PRs in the ONL beginning at postnatal day (P) 11, until complete degeneration is reached by P28, at which just a single row of PR nuclei remain. Similarly, S334ter-3 rat retinas showed near full ONL thickness at P14 (FIG. 10A, bracket), compared with a single discontinuous row of remaining PR nuclei at P33 (FIG. 10C, bracket). As previously described for line-4 S334 rats, RHO Nterminal immunolabel identified both the truncated (RHO_{S334}) and full length (RHO_{WT}) isoforms. However, RHO C-terminal specific immunolabel exclusively identified the RHO_{WT} isoform. Immunolabeled N-terminal RHO in P14 S334ter-3 retinas was predominantly mislocalized at PR cell bodies throughout the ONL, while RHO_{WT} was present in the ONL and polarized toward PR inner segments (IS) (FIG. 10A vs. FIG. 10B). The consequence of RHO mistrafficking by P33 was the degeneration of PRs in the ONL, which consisted of a single discontinuous layer of PR nuclei (FIG. 10C, FIG. 10D).

### Example 15

### gRNA Vector Design & Strategy

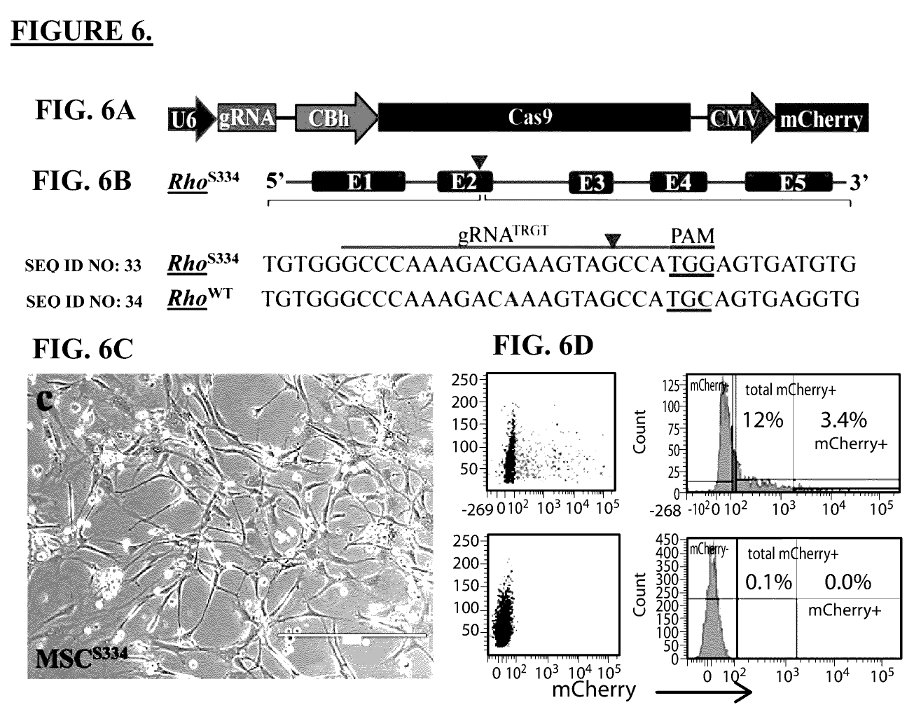

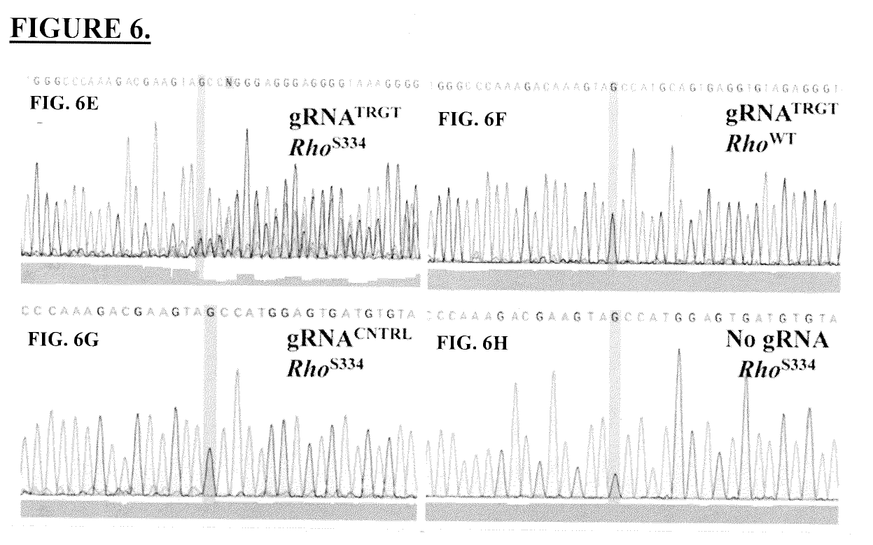

The Inventors designed a 20 nucleotide targeting-gRNA construct (gRNA_{TRGT}) complementary to a region in exon 1 immediately upstream of a PAM unique to the Rho_{S334} locus in order to discriminate alleles during Cas9 cleavage (FIG. 6A, FIG. 6B). The homology between mutant and WT Rho alleles at the gRNA binding locus differs by one nucleotide at position 10/20 (FIG. 6B). gRNA_{TRGT} targeted the second of four PAMs identified as unique to Rho_{S334} to give the greatest probability for allele-specific ablation. The Inventors reasoned that 3′ open reading frame disruptions downstream to the two PAMs in exon 4 risked generating further truncated RHO isoforms, while targeting the 5′-most PAM of exon 1 risked Rho_{S334} ORF restoration as a consequence of polymerase skipping. While the S334ter mutation was not the target of the Inventors' gRNA_{TRGT}, dominant adRP mutations have been identified in patients that generated novel PAM sequences that may be targetable similar to the current strategy (Table 1).

### Example 16

### Rho_{S334} Disruption In Vitro

Rho_{S334}-selective cleavage with gRNA_{TRGT} was confirmed in cultured bone marrow-derived stromal cells derived from S334ter-3 rats (MSC_{SS334}). mCherry+ MSC_{SS334} were isolated by fluorescence activated cell sorting (FACS) 3 days after transfection (FIG. 6C, FIG. 6D), and PCR-amplified regions of genomic DNA that encompassed the predicted Cas9 cleavage sites were sequenced. Multiple peaks and decreased nucleotide read fidelity (Phred quality scores) indicated the presence of novel genomic sequences originating downstream from the disparate PAM of Rho_{S334}, which was absent at the homologous Rho_{WT} locus (FIG. 6E, FIG. 6F). Genomic disruption was not observed using a second gRNA that targeted exon 2 of Rho_{S334}, which had four nucleotide mismatches from Rho_{WT}, and was thus used as a control gRNA (gRNA_{CNTRL}) for in vivo experiments (FIG. 6G). Likewise, genomic disruption was not observed in cells from untreated retinas (FIG. 6H).

### Example 17

### Rho_{S334} Disruption In Vivo

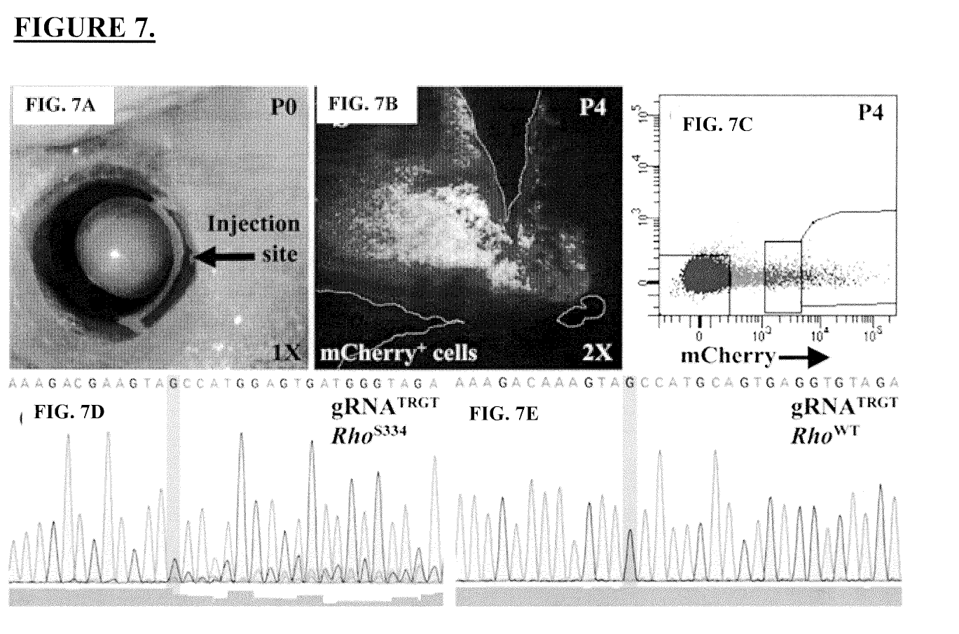

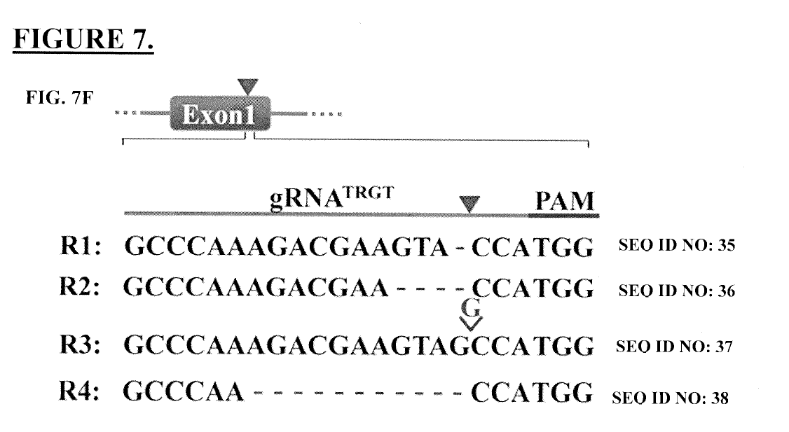

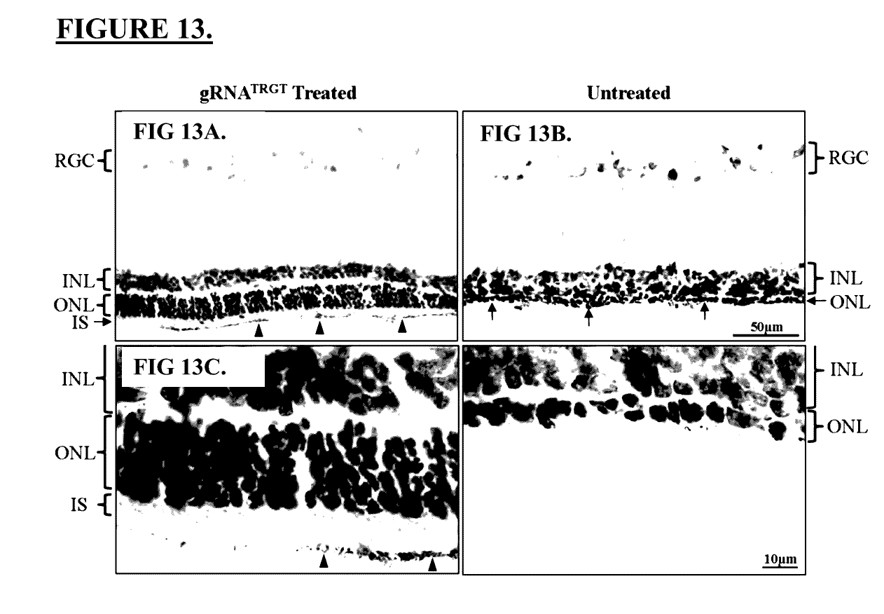

To evaluate Rho_{S334} disruption in vivo, S334ter-3 rats received a single unilateral subretinal injection of the gRNA_{TRGT} construct at P0 (FIG. 7A), immediately followed by electroporation in order to facilitate plasmid uptake by PRs in cell-cycle. Retinas were dissociated at P4 and plasmid-transfected cells were sorted on the basis of mCherry expression (FIG. 7B, FIG. 7C), followed by genomic DNA extraction for Sanger sequencing. The Inventors confirmed multiple genomic sequences resulting from Rho_{S334}-selective cleavage downstream the Cas9 cleavage site (blue highlight) in P4 mCherry+ PRs (FIG. 7D), but no cleavage was detected at the Rho_{WT} locus (FIG. 6E). Genomic cleavage was similar in PRs that expressed mCherry at high (mCherry_{Hi}) or intermediate/low (mCherry_{Lo}) levels in three rats (FIG. 12). PCR amplified Genomic DNA regions encompassing the gRNA_{TRGT} target site were further sequenced after clonal expansion in NEB-á to determine indel frequency. The ratio of clones in which indels were detected from two rats represented cleavage efficiencies of 33% (7/21 clones) and 36% (15/42 clones). Indels were not detected at the Rho_{WT} locus, or the next 8 motif mismatch-predicted off-target loci (Table 2). Similarly, histological evaluation using cresyl violet stain showed phenotypic rescue by gRNA_{TRGT} treatment compared with the contralateral untreated eye of the same animal (FIG. 13A, FIG. 13C vs. FIG. 13B, FIG. 13D)

### Example 18

### Photoreceptor Preservation Following Rho_{S334} Ablation

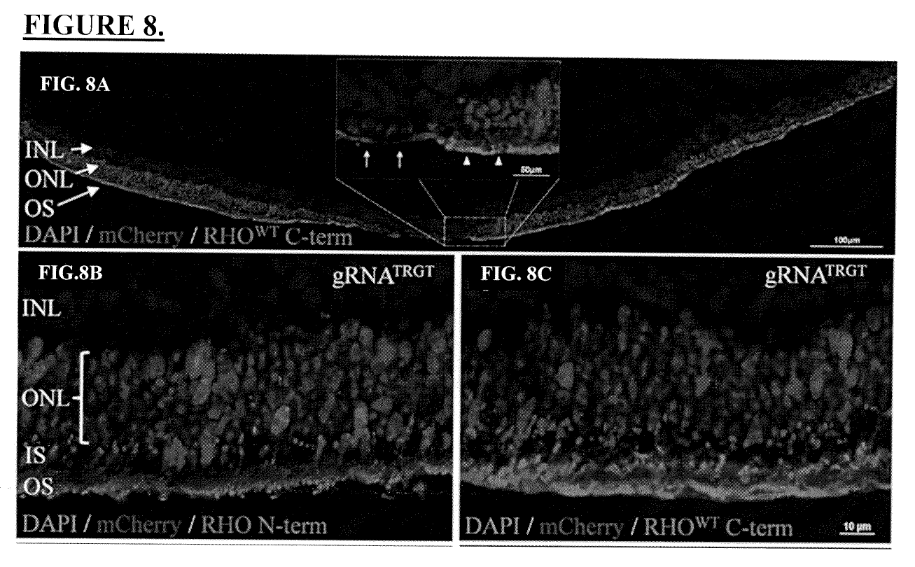

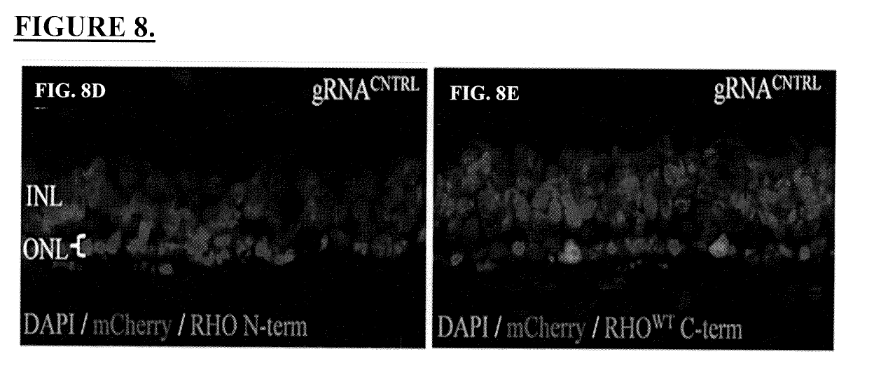

To determine whether the retinal phenotype was altered in S334ter-3 rats, gRNA_{TRGT} and gRNA_{CNTRL} treatments were assessed by immunohistology at P33. Extensive and robust retinal preservation was observed in gRNA_{TRGT}-injected eyes with up to 8 layers of rescued PRs, in sharp contrast to the single PR layer in gRNA_{CNTRL}-treated retinas (FIG. 8A-FIG. 8C vs. FIG. 8D, FIG. 8E). PR rescue was observed exclusively in transfected regions demonstrated by coincident mCherry expression, which were the only regions with observable POS formation, morphologically characteristic of the WT-phenotype that was absent in gRNA_{CNTRL}-injected eyes (FIG. 8D, FIG. 8E). Furthermore, N-terminal RHO labeling confirmed the absence of RHO_{S334} from PR cell bodies in the rescued ONL (FIG. 8B), while C-terminal labeling showed RHO_{WT} to be strictly confined to POS (FIG. 8C). These data suggest that Rho_{S334} was ablated and the toxic effect of RHO_{S334} was removed in transfected regions, which subsequently permitted proper RHO_{WT} trafficking, POS formation, and PR survival. gRNA_{CNTRL}-treated retinas lacked appreciable amounts of either RHO isoform at P33 due to extensive degeneration (FIG. 8D, FIG. 8E).

### Example 19

### Retinal Synapse Preservation Following Rho_{S334} Ablation

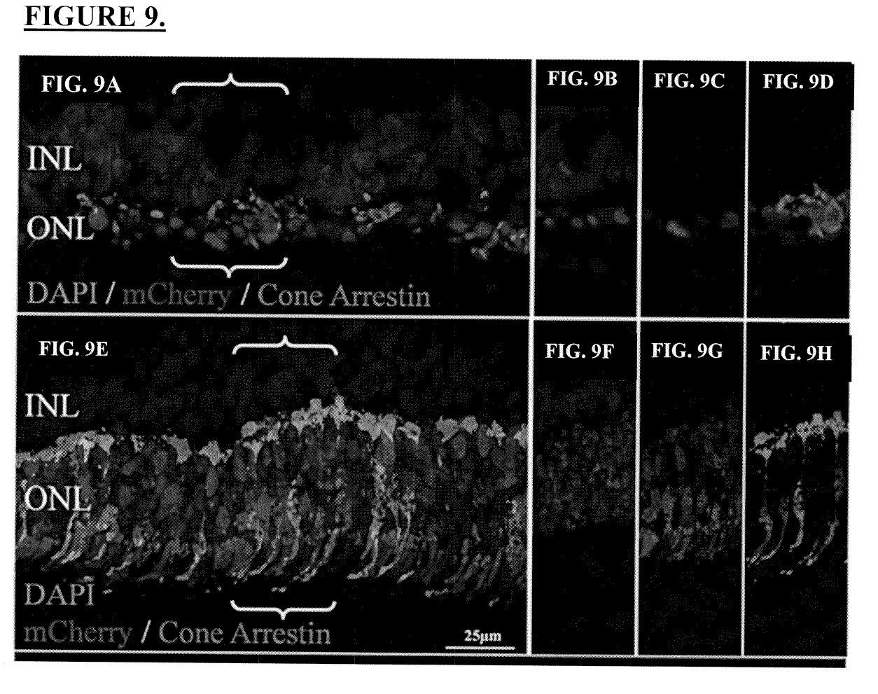

Immunostaining of degenerated gRNA_{CNTRL}-treated retinas revealed that the single layer of remaining PRs were non-Rho-expressing cone cells (FIG. 9A, FIG. 9D), the morphology of which was well-preserved with gRNA_{TRGT} treatment (FIG. 9E, FIG. 9H). mCherry fluorescence intensity in PRs appeared highest in mitochondria-rich inner segments (Figure FIG. 9G). In addition, gRNA_{TRGT} treatment preserved the dendritic arborization of rod-bipolar cells (PKC-α, FIG. 9I) and synaptic density between second-order inner retinal neurons and PRs within rescued retinal areas (synaptophysin; FIG. 9J), compared with adjoining degenerated areas.

### Example 20

### Quantification of PR Density and Visual Function in Rho_{S334} Treated Retinas

The extent of mCherry distribution in retinal flat-mounts at P33 reached 29% maximal area coverage, with uneven fluorescence intensity in transfected regions (FIG. 10A). By cross section analysis, the density of PRs in mCherry+ regions from gRNA_{TRGT} treatment at P33 was 307±82 PR nuclei/100 μm, or 9-fold greater than from gRNA_{TRGT} treatment (33±3, nuclei/100 μm P<0.002), and was similarly higher when compared with comparable regions (non-fluorescent) of untreated control (27±13 nuclei/100 μm, P<0.001). The density of PR nuclei was not different between gRNA_{CNTRL} and untreated groups (P>0.41) (FIG. 10B). Visual acuity assessed at P39 by optokinetic response (OKR) was 53% higher from gRNA_{TRGT} compared with that of gRNA_{CNTRL} treatment (0.185 versus 0.121 cycles/degree, respectively; FIG. 10C). By using the contralateral eye of individual animals as internal controls, visual acuity was 35% higher in the gRNA_{TRGT}-treated eye compared with the fellow eye in individual animals, whereas, gRNA_{CNTRL}-injection reduced visual acuity by 2.3% compared to the contralateral untreated eye (FIG. 10D).

### Example 21

### Dual AAV Vectors

The above results demonstrate that photoreceptors are rescued following in vivo RhoS334ter ablation by injection of Cas9/gRNA plasmid into S334ter rats at P0. The S. Pyogenes Cas9 transcript maybe too large (currently) to be packaged into AAV particles, delivered here via which necessitated in vivo electroporation or injection of plasmid DNA at postnatal day 0. The smaller transcript of the S. aureus Cas9 allows packaging into AAV particles for translational in vivo delivery at postnatal day 15. One approach is to rely on smaller SaCas9 constructs to be packaged into AAV particles in which the RhoS334ter transgene is targeted in S334ter-3 rats. Alternatively, if Cas9/gRNA cannot be delivered by electroporation or injection. Due to the size limitation of AAV particles (˜4.7 kb), we propose to simultaneously inject two AAV vectors; AAV-Cas9 and AAV-gRNA with a fluorescent reporter as previously described.

### Example 22

### Eosomes

In collaboration with Dr. Lali Medina-Kauwe. Eosomes are 10 nm-20 nm self-assembling modular peptides with an N-terminal targeting peptide composed of the adenoviral penton base, a central membrane penetration peptide, and a C-terminus capturing peptide onto which therapeutic peptides/nucleic acids can be loaded. The modular core protein can adapted for PR-specific delivery. Eosomes specifically deliver the therapeutic payload to the interior of a target cell avoiding lysosomal degradation. For CRISPR/Cas9 plasmid delivery, DNA condensation will be achieved using protamine sulfate treatment. h

### Example 22

### Peptide for Ocular Delivery (POD)

A small (3.5 Kd) peptide that can bind the cell membrane and enter the cytoplasm within 5 minutes in culture and within 2 hours in tissues in vivo. This peptide for ocular delivery (POD) can be used to deliver small fluorophores such as lissamine into retinal pigment epithelium (RPE), photoreceptors, ganglion cells etc. One can envisage replacing the lissamine with small molecule drugs. http://emerald.tufts.edu/˜rkumar02/RKSLabWebsite/POD.html

### Example 23

### Liposomes

Liposome-mediated delivery of the Cas9 protein will be tested. The decreased latency for endonuclease activity compared with plasmid expression will promote rapid gene editing in the fast degeneration S334ter rat model and minimize the potential for off-target cleavage events. Pre-assembled gRNA and Cas9 protein complexes (including a Nuclear Localization Sequence NLS) is commercially available as an injection-ready formulation, which we will test by subretinal injection. There is evidence supporting efficient transfection of exogenous proteins into eyes of adult rodents using a charged liposome (Pep1/Chariot, intracellular delivery of proteins into mouse Müller glia cells, Chariot™ protein delivery reagent). Furthermore, as transcription and translation of plasmid DNA is unnecessary for the Cas9 protein after transfection, DNA cleavage begins within 2 hours, a time-frame faster than the current electroporation method (8 hrs). As this strategy precludes the use of fluorescent reporters, we will determine Cas9 distribution within the subretinal space by labeling Cas9 with FITC according to published protocols. Retinas will be imaged by live in vivo imaging using a Micron III to determine distribution and longevity of fluorescent signal. For some experiments, the Cas9/gRNA complex will be pre-combined with unconjugated mouse IgG_{2A} antibody for subsequent immunohistochemical analysis.

### Example 24

### Discussion

These data collectively provide proof of principle for in vivo allele-specific ablation using CRISPR/Cas9 to prevent inherited retinal degeneration. The selective ablation of Rho_{S334} had prevented RHO_{S334} accumulation at PR cell bodies in the ONL and restored RHO_{WT} trafficking to outer segments, which prevented retinal degeneration and preserved visual acuity. Eliminating RHO_{S334} expression generated blunt transition areas at which markedly preserved retinal areas adjoined those with advanced degeneration. These transition regions likely represent the physical extent to which gRNA_{TRGT} transfection had reached, as mCherry expression shared the same demarcation (FIG. 9A, and FIG. 9I, FIG. 9J). The observation that mCherry expression was not observed in all rescued PRs is likely attributable to the episomal nature of the plasmids and the reduced efficiency of the CMV promoter (driving mCherry) compared to that of Chicken â-Actin-derivative (driving Cas9). Thus, mCherry expression likely underestimated the total number of PRs in which Cas9/gRNA_{TRGT} was active and therefore the number of PRs in which the Rho_{S334} ablation-mediated rescue had occurred. This assertion is consistent with the S334ter-3 model, in which the expression of Rho_{S334} is exclusive to (rod) PRs and instigates cell-autonomous apoptotic signaling, suggesting that Rho_{S334} ablation is the only manner by which phenotypic rescue could have been achieved with gRNA_{TRGT} treatment. Furthermore, since PRs possess a single copy of the Rho_{S334} transgene, its ablation is absolute for conferring PR survivability. Whether this permanent genomic change confers long-term PR survival in a degenerative milieu will require sequential analysis of gRNA_{TRGT}-treated retinas. Toward this goal, PR loss was shown to be halted in an inducible transgenic model of autosomal recessive RP, even at advanced stages of degeneration. Furthermore, a translational proof-of-concept study using gene replacement in a canine model of X-lined RP demonstrated the feasibility of long-term arrest of PR loss.₁₈ These studies support the possibility of long-term vision rescue following in vivo gene correction. With regard to visual function assessment, optomotor reflexes were significantly preserved from a single gRNA_{TRGT} treatment 39 days prior, compared with controls. Differences in visual function were not detected by electroretinography (ERG), however, ERG sensitivity is limited for detecting focal retinal activity and may not represent an appropriate test for the Inventors' experimental approach. Translational limitations of the current methods are technical in nature and greater functional improvement may be obtained through alternative methods to maximize retinal transfection and genetic correction. For example, the use of shorter Cas9 orthologs, such as Staphylococcus aureus (˜3.3 kb) with short universal tRNA promotor₂₁ will allow for efficient vector packaging into adeno-associated viral particles. Alternatively, direct delivery of Cas9 protein/gRNA complexes in vivo would also minimize the duration of endonuclease activity and therefore the risk for off-target cleavage events without compromising targeted cleavage efficiency, a critical consideration in the context of using genomic ablation as therapy.

The first in vivo functional correction of an inherited dominant mutation using CRISPR, shown here, provides proof-of-concept that CRISPR/Cas9 can be used to treat inherited adRP. Selective-ablation of a dominant allele was achieved by targeting a PAM unique to the Rho_{S334} transgene, which differed from the Rho_{WT} sequence by a single nucleotide. adRP-linked missense mutations that likewise create targetable monoallelic PAM sequences have been identified in patients (Table 1), who may thus represent the candidate population for ablation therapy to achieve phenotypic rescue. The challenge of generating targeted therapies for diseases with mutational heterogeneity may be addressed by altering the PAM specificity of Cas through rational-design engineering or by using non-canonical Cas enzymes. Such efforts may broaden the number of targetable mutations, and thereby expand the treatable pool of patients with degenerative diseases of the retina, and possibly other tissues.

TABLE 1
Nucleotide Change

| | Pheno- | a.a. | Nucleotide |
|---|---|---|---|
| Gene | type | Substitution | Change |
| RHO | ADRP | Pro 347 Arg | CCG→CGG |
| | ADRP | Met 207 Arg | ATG→AGG |
| | ADRP | Met 216 Arg | ATG→AGG |
| | ADRP | Asp 190 Gly | GAC→GGC |
| | ADRP | Ser 186 Trp | TCG→TGG |
| | ADRP | Cys 167 Trp | TGC→TGG |
| | ADRP | Thr 58 Arg | ACG→AGG |
| | ADRP | Leu 46 Arg | CTG→CGG |
| | ADRP | 998 Ins-4 bp | GCC TCT→GCA |
| | | | GGC CTC T |
| | | | [SEQ ID NO: 1] |
| RPGR | RP3 | Arg 127 Gly | AGA-GGA |
| | RP3 | Arg 780 Arg | AGA-AGG |
| | RP3 | 1572IVS13+11A/G | TGAAG→TGGAG |
| | RP3 | 237 Del-AT | AGA TGT→AG--GT |
| | RP3 | 1573IVS13-2A-G | TACAG AAA→TAC |
| | | | GGAAA ] |
| PRPH | ADRP | Lys 153 Arg | AAG→AGG |
| | ADRP | Ser 212 Gly | AGC→GGC |
| | RP | Gln 178 Arg | CAG→CGG |
| RDH | Polymorphism | Gln 161 Arg | CAG→CGG |
| | CSRD | Ser 175 Pro | TCG→TGG |
| CRX | | 742 Ins 23 bp | CC→CTCCGTGGGAC |
| | | | CTTCCCTGGCCCC |
| | | | [SEQ ID NO: 2] |
| ROM1 | Polymorphism | Ala 118 Gly | GCC→GGC |

TABLE 2

| | | | | | |
|---|---|---|---|---|---|
| | Genome | | | | Cleavage |
| Rho | Target | Forward | Reverse | Rho | Efficiency |
| TRGT | Site | Priming Site | Priming Site | Cleavage | % |

Off-target cleavage sites RhoWT identified by
motif-mismatch prediction and On-target efficiency

| | | | | | |
|---|---|---|---|---|---|
| OT1.1 | RhoWT Locus | GGCGTGGTGCGCAGC | ACCCATTAAGCTGCT | 0/11 | 0 |
| | (rat #6) | CCCTTT | GCTTCC | | |
| | | [SEQ ID NO: 3] | [SEQ ID NO: 4] | | |
| OT1.1 | RhoWT Locus | GGCGTGGTGCGCAGC | ACCCATTAAGCTGCT | 0/19 | 0 |
| | (rat #8) | CCCTTT | GCTTCC | | |
| | | [SEQ ID NO: 5] | [SEQ ID NO: 6] | | |
| OT1.2 | >chromosome: | CAAAAGGCCAAACTG | ATTTGGCTGCGTCTG | 0/10 | 0 |
| | Rnor_5.0:17: | CCCTAAG | TAGATGA | | |
| | 7716236: | [SEQ ID NO: 7] | [SEQ ID NO: 8] | | |
| | 7716852:-1 | | | | |
| OT1.3 | >chromosome: | TGGTTGTTGGGTACT | GGCGCGCATAGTTGC | 0/18 | 0 |
| | Rnor_5.0:2: | GGTAGATG | AGTTT | | |
| | 184344617: | [SEQ ID NO: 9] | [SEQ ID NO: 10] | | |
| | 184345231:1 | | | | |
| OT1.4 | >chromosome: | CTTGGCCGAGGTACT | TTCAGTTTCCTGATG | 0/11 | 0 |
| | Rnor_5.0:2: | TCTCA | GCAATGTA | | |
| | 170133015: | [SEQ ID NO: 11] | [SEQ ID NO: 12] | | |
| | 170133631:-1 | | | | |
| OT1.5 | >chromosome: | ACTCAATTCCCCAAT | TGTGAGGTCCAACAT | 0/10 | 0 |
| | Rnor_5.0:7: | AAAAAGGCA | GTGCT | | |
| | 4806038: | [SEQ ID NO: 13] | [SEQ ID NO: 14] | | |
| | 4806653:-1 | | | | |
| OT1.6 | >chromosome: | TCCACGAACAGCCAA | GTTGCCAGCCTAGAT | 0/10 | 0 |
| | Rnor_5.0:17: | CCTT | AACCTCA | | |
| | 60638647: | [SEQ ID NO: 15] | [SEQ ID NO: 16] | | |
| | 60639262:-1 | | | | |
| OT1.7 | >chromosome: | CCTCCTATGTAGAAC | GGTGGATCACCTATC | 0/10 | 0 |
| | Rnor_5.0:4: | TGGCTCA | TGGTTC | | |
| | 54769077: | [SEQ ID NO: 17] | [SEQ ID NO: 18] | | |
| | 54769691:1 | | | | |
| OT1.8 | >chromosome: | GCTTCTCTCTGGTTG | AGGCTGCTGCTCCAG | 0/13 | 0 |
| | Rnor_5.0:8: | TGGCT | ATTTT | | |
| | 96494989: | [SEQ ID NO: 19] | [SEQ ID NO: 20] | | |
| | 96495603:1 | | | | |
| OT1.9 | >chromosome: | GGCTGGTGATGGTGG | AACAGTGTCTTCCAG | 0/11 | 0 |
| | Rnor_5.0:1: | TCATT | CTGCC | | |
| | 215724446: | [SEQ ID NO: 21] | [SEQ ID NO: 22] | | |
| | 215725060:-1 | | | | |

On-target efficiency in Rho^{S334}

| | | | | | |
|---|---|---|---|---|---|
| gRNA | RhoS334 Locus | AACTTCCTCACGCTC | CCAATGAATAAGCTG | 7/21 | 33 |
| S334 | (rat #6) | TACGTC | GGCCT | | |
| | | [SEQ ID NO: 23] | [SEQ ID NO: 24] | | |
| gRNA | RhoS334 Locus | AACTTCCTCACGCTC | CCAATGAATAAGCTG | 15/42 | 36 |
| S334 | (rat #8) | TACGTC | GGCCT | | |
| | | [SEQ ID NO: 25] | [SEQ ID NO: 26] | | |

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the compositions for, and methods of, genetic editing, in vivo methods associated with genetic editing, compositions of cells generated by the aforementioned techniques, treatment of diseases and/or conditions that relate to the teachings of the invention, techniques and composition and use of solutions used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention. Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of treatment comprising:
(a) providing a quantity of one or more therapeutic vectors; and
(b) administering the one or more therapeutic vectors to a mammal afflicted with a disease and/or condition, wherein in vivo expression of the one or more therapeutic vector is capable of treating the mammal for the disease and/or condition.

2. The method of claim 1, wherein the one or more therapeutic vectors, each encode at least one clustered regularly interspaced short palindromic (CRISPR) protein and one or more guide RNAs (gRNAs).

3. The method of claim 2, wherein the CRISPR protein is cas9.

4. The method of claim 1, wherein the one or more gRNAs comprise a sequence cognate to a target polynucleotide sequence and capable of binding to a protospacer adjacent motif (“PAM”).

5. The method of claim 4, wherein the PAM comprises the sequence NGG or NNGRRT.

6. The method of claim 1, wherein the disease and/or condition comprises a dominant mutation.

7. The method of claim 6, wherein the disease and/or condition comprising a dominant mutation is retinitis pigmentosa (RP).

8. The method of claim 7, wherein the RP comprises a mutation in rhodopsin (RHO).

9. The method of claim 1, wherein the mammal comprises a human.

10. The method of claim 1, wherein the therapeutic vector comprises an adenovirus, adeno associated virus or lentivirus.

11. The method of claim 1, wherein administering the one or more therapeutic vectors comprises subretinal injection.

12. The method of claim 1, wherein treating the mammal for the disease and/or condition comprises in vivo generation of a double stranded break in a population of cells in the mammal.

13. The method of claim 1, further comprising providing a quantity of DNA template in step (a) and co-administering the DNA template in step (b).

14. The method of claim 1, wherein the disease and/or condition comprises a recessive mutation.

15. An in vivo method of genomic editing comprising:
(a) providing a quantity of one or more vectors each encoding at least one clustered regularly interspaced short palindromic (CRISPR) protein and one or more guide RNAs (gRNAs); and
(b) administering the one or more vectors to a mammal, wherein in vivo expression of the one or more vectors comprises binding of the CRISPR protein to a locus cognate to the gRNA and in vivo generation of a double stranded break (DSB) in a population of cells in the mammal, wherein in vivo homologous recombination (HR) of the DSB results in editing of the genome of a population of cells in the mammal.

16. The method of claim 15, wherein the CRISPR protein is cas9 and the one or more gRNAs comprise a sequence capable of binding to a protospacer adjacent motif (“PAM”).

17. The method of claim 15, wherein HR comprises non-homologous end joining (NHEJ) introducing missense or nonsense of a protein expressed at the locus.

18. The method of claim 15, wherein HR comprises homology directed repair (HDR) introducing template DNA co-administered in step (b).

19. The method of claim 15, wherein the HR corrects a dominant mutation.

20. The method of claim 15, wherein the HR corrects a recessive mutation.

21. The method of claim 15, wherein the vector comprises an adenovirus, adeno associated virus or lentivirus.

22. The method of claim 19, wherein the dominant mutation comprises a mutation in rhodopsin (RHO), the mammal comprises a human, and administering the one or more vectors comprises subretinal injection.

23. A composition comprising:
a vector encoding a clustered regularly interspaced short palindromic (CRISPR) protein and one or more guide RNAs (gRNAs), wherein the one or more gRNAs comprise a sequence cognate to a target polynucleotide sequence and capable of binding to a protospacer adjacent motif (“PAM”).

24. The composition of claim 23, wherein the CRISPR protein is cas9 and the gRNA is cognate to a locus encoding rhodopsin (RHO).
